(19) United States
(12) Patent Application Publication
(10) Pub. No.: US 2005/0240239 A1
(43) Pub. Date: Oct. 27, 2005

(54) Method and system for gastric ablation and gastric pacing to provide therapy for obesity, motility disorders, or to induce weight loss

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/169,468

(22) Filed: Jun. 29, 2005

(51) Int. Cl. A61N 1/18

(52) U.S. Cl. 607/40

(57) ABSTRACT

Method and system to provide therapy for obesity, gastric motility, or to induce weight loss comprises ablating the gastric tissue around the “pacemaker” region of the stomach, and electrically pacing the stomach with a pulse generator/stimulator to control the electrical activity of the gastric muscle. The ablation to the gastric tissue may be from the epigastric side, or may be from inside the stomach. The ablation may be performed utilizing any one of: radiofrequency catheter ablation; radiofrequency catheter ablation using an irrigated tip catheter; microwave ablation; cryoablation; high intensity focused ultrasound (HIFU) ablation; and laser ablation. The ablation of the “pacemaker” region of the stomach may be partial or complete. A gastric pulse generator/stimulator is implanted to provide electrical pulses to the stomach. The function of the gastric stimulator after complete ablation of the pacemaker region, is to provide a basic electrical rhythm (BER) to regulate and control electrical activity of the stomach. Alternatively, if partial ablation is performed the function of the gastric pulse generator/stimulator is to enhance the residual basic electrical rhythm (BER), or to interfere with the residual basic electrical rhythm (BER).

This application is related to a co-pending application entitled “Gastrointestinal (GI) ablation for GI tumors or to provide therapy for obesity, motility disorders, GERD, or to induce weight loss” filed Jun. 29, 2005.

### FIELD OF INVENTION

This invention relates generally to medical ablation and pacing, more specifically to stomach wall ablation and gastric pacing to provide therapy for obesity, motility disorders, or to induce weight loss.

### BACKGROUND

Obesity is a significant health problem in the United States and many other developed countries. Obesity results from excessive accumulation of fat in the body. It is caused by ingestion of greater amounts of food than can be used by the body for energy. The excess food, whether fats, carbohydrates, or proteins, is then stored almost entirely as fat in the adipose tissue, to be used later for energy. Obesity is not simply the result of gluttony and a lack of willpower. Rather, each individual inherits a set of genes that control appetite and metabolism, and a genetic tendency to gain weight that may be exacerbated by environmental conditions such as food availability, level of physical activity and individual psychology and culture. Other causes of obesity include psychogenic, neurogenic, and other metabolic related factors.

Obesity is defined in terms of body mass index (BMI), which provides an index of the relationship between weight and height. The BMI is calculated as weight (in Kilograms) divided by height (in square meters), or as weight (in pounds) times 703 divided by height (in square inches). The primary classification of overweight and obesity relates to the BMI and the risk of mortality. The prevalence of obesity in adults in the United States without coexisting morbidity increased from 12% in 1991 to 17.9% in 1998.

Treatment of obesity depends on decreasing energy input below energy expenditure. Treatment has included among other things various drugs, starvation and even stapling or surgical resection of a portion of the stomach. Surgery for obesity has included gastroplasty and gastric bypass procedure. Gastroplasty which is also known as stomach stapling, involves constructing a 15- to 30 mL pouch along the lesser curvature of the stomach. A modification of this procedure involves the use of an adjustable band that wraps around the proximal stomach to create a small pouch. Both gastroplasty and gastric bypass procedures have a number of complications.

This Application is directed to providing therapy or alleviating symptoms for obesity and other gastrointestinal (GI) disorders, by ablating the pacemaker region of the stomach and electrically pacing the stomach at a rate which is appropriate for achieving the desired effect, for the particular disorder. In this disclosure, the terms stomach, stomach muscle, gastric wall, and gastric wall muscle are used interchangeably.

The ablations to the pacemaker region may be performed from the epigastric side via laproscopic surgery. Alternatively, catheter ablations may be performed on the endogastric side via the mouth and esophagus. The ablation technology may be one from a group comprising:

a) Radiofrequency catheter ablation;

b) Radiofrequency ablation using irrigated tip catheter;

c) Microwave ablation;

d) Cryoablation;

e) High intensity focused ultrasound (HIFU) ablation; and

f) Laser ablation.

Gastric pacing may be performed utilizing an implantable pulse generator (IPG), a rechargeable implantable pulse generator, or an external stimulator utilizing an implanted stimulus-receiver. Currently available cardiac pacemakers and nerve stimulators can also be adapted for gastric pacing.

### Background of Gastrointestinal (GI) Physiology and Regulation

Shown in conjunction with FIG. 1, the gastrointestinal (GI) tract is a continuous muscular digestive tube that winds through the body. The organs of the GI tract are the mouth, pharynx (not shown), esophagus 3, stomach 54, small intestine (duodenum 7, jejunum, and ileum), and large intestine (cecum, ascending colon, transverse colon, and descending colon).

The gastrointestinal (GI) tract has a nervous system all its own, which is the enteric nervous system 9. This is shown in conjunction with FIG. 2. It lies entirely in the wall of the gut, beginning in the esophagus 3 and extending all the way to the anus. The enteric nervous system has about 100 million neurons, almost exactly equal to the number in the entire spinal cord. It especially controls gastrointestinal movements and secretion. The enteric nervous system is composed mainly of the two plexuses, 11) the myenteric plexus 10, which is the outer plexus lying between the longitudinal and circular muscle layers, and 2) the submucosal plexus 11 that lies in the submucosa. The nervous connection within and between these two plexuses is depicted in FIG. 2. The myenteric plexus controls mainly the gastrointestinal movements, and the submucosal plexus controls mainly gastrointestinal secretion and local blood flow. As also depicted in FIG. 2, the sympathetic and parasympathetic fibers connect with the myenteric 10 and the submocosal 11 plexus. Although the enteric nervous system can function on its own, stimulation by the parasympathetic 12 and sympathetic 13 systems can further activate or inhibit gastrointestinal functions. The autonomic nerves influence the functions of the gastrointestinal tract by modulating the activities of neurons of the enteric nervous system 9.

Shown in conjunction with FIGS. 2, sympathetic innervation of the gastrointestinal tract is mainly via postganglionic adrenergic fibers whose cell bodies are located in pre-vertebral and parabertabral ganglia. The celiac, superior and inferior mesenteric, and hypogastric plexus provide sympathetic innervation to various segments of the GI tract. Activation of the sympathetic nerves usually inhibits the motor and secretory activities of the GI system.

Parasympathetic innervation of the GI tract down to the level of the transverse colon is provided by branches of the vagus nerves (10^{th} cranial nerve). Excitation of parasympathetic nerves usually stimulates the motor and secretory activities of the GI tract.

The stomach 54 is richly innervated by extrinsic nerves and by the neurons of the enteric nervous system 9. Axons from the cells of the intramural plexus innervate smooth muscle and secretory cells.

The emptying of gastric contents is regulated by both neural and hormonal mechanisms. The duodenal and jejunal mucosa contain receptors that sense acidity, osmotic pressure, certain fats and fat digestion products, and peptides and amino acids The chyme that leaves the stomach is usually hypertonic and it becomes even more hypertonic because of the action of the digestive enzymes in the duodenum. Gastric emptying is slowed by hypertonic solutions in the duodenum, by duodenal pH below 3.5, and by the presence of amino acids and peptides in the duodenum, The presence of fatty acids or monoglycerides (products of fat digestion) in the duodenum also dramatically decreases the rate of gastric emptying.

Parasympathetic innervation to the stomach is supplied by the vagus nerves, while sympathetic innervation to the stomach is provided by the celiac plexus. In general, parasympathetic nerves stimulate gastric smooth muscle motility and gastric secretions, whereas sympathetic activity inhibits these function. Numerous sensory afferent fibers leave the stomach in the vagus nerves; some of these fibers travel with sympathetic nerves. Other sensory neurons are the afferent links between sensory receptors and the intramural plexuses of the stomach. Some of these afferent fibers relay information intragastric pressure, gastric distention, intragastric pH, or pain.

Shown in conjunction with FIG. 3 is the fundus 15, the body 17, and antrum 19 of the stomach 54. After eating, when a wave of esophageal peristalsis begins, a reflex causes the LES to relax. This relaxation of the LES is followed by receptive relaxation of the fundus 15 and body 17 of the stomach. The stomach 54 will also relax if it is filled directly with gas or liquid. The nerve fibers in the vagi are a major efferent pathways for reflex relaxation of the stomach 54.

FIG. 4 depicts the three main muscle layers of the stomach 54, which are the longitudinal layer 14, the circular layer 16, and the oblique layer 18. The complex and coordinated activity of these muscle layers is responsible for the normally efficient gastric motility. Whereas, the gastric pacing disclosed here from around the antral area of the stomach 54, disrupts the normal gastric motility.

Normally, the smooth muscle of the GI tract is excited by almost continual slow, intrinsic electrical activity along the membranes of the muscle fibers. This activity has two basic types of electrical waves: 1) slow waves and 2) spikes. This is shown in conjunction with FIG. 5. Most gastrointestinal contractions occur rhythmically, and this rhythm is determined mainly by the frequency of the slow waves of the smooth muscle membrane potential. Their intensity usually varies between 5 and 15 millivolts, and their frequency ranges in different parts of the human gastrointestinal tract between 3 and 12 per minute. The rhythm of contraction of the body of the stomach is about 3 per minute (and in the duodenum is about 12 per minute).

The electrical activity of the GI tract is shown in conjunction with FIG. 5. For example, the contraction of small intestinal smooth muscle occurs when the depolarization caused by the slow wave exceeds a threshold for contraction. When depolarization of a slow wave exceeds the electrical threshold, a burst of action potentials 19 occurs. The action potentials elicit a much stronger contraction than occurs in the absence of action potentials. The contractile force increases with increasing number of action potentials.

Action potentials in gastrointestinal smooth muscle are more prolonged (10 to 20 msec) than those of skeletal muscle and have little or no overshoot. The rising phase of the action potentials is caused by ion flow through channels that conduct both Ca^{++ and Na}⁺and are relatively slow to open. Ca++ that enters the cell during the action potential helps to initiate contraction.

When the membrane potential of gastrointestinal smooth muscle reaches the electrical threshold, typically near the peak of a slow wave, a train of action potentials (1 to 10/sec) is fired. The extent of depolarization of the cells and the frequency of action potentials are enhanced by some hormones and paracrine agonists and by compounds liberated from excitatory nerve endings. Inhibitory hormones and neuroefector substances hyperpolarize the smooth muscle cells and may diminish or abolish action potential spikes.

Slow waves that are not accompanied by action potentials elicit weak contractions of the smooth muscle cells (FIG. 5). Much stronger contractions are evoked by the action potentials that are intermittently triggered near the peaks of the slow waves. The greater the frequency of action potentials that occur at the peak of a slow wave, the more intense is the contraction of the smooth muscle. Because smooth muscle cells contract rather slowly (about one tenth as fast as skeletal muscle cells), the individual contraction caused by each action potential in a train do not cause distinct twitches; rather, they sum temporally to produce a smoothly increasing level of tension (FIG. 5).

Between trains of action potentials the tension developed by gastrointestinal smooth muscle falls, but not to zero. This nonzero resting, or baseline, tension of smooth muscle is called tone. The tone of gastrointestinal smooth muscle is altered by neuroeffectors, hormones, paracrine substances, and drugs.

Control of the contractile and secretory activities of the gastrointestinal tract involves the central nervous system, the enteric nervous system, and hormones and paracrine substances. The autonomic nervous system typically only modulates the patterns of muscular and secretary activity; these activities are controlled more directly by the enteric nervous system.

In the current invention, ablation of the stomach is performed at the pacemaker zone of the stomach. By ablating at, and around the pacemaker region, the intent is to decrease basic electrical rhythm (BER), whereby the stomach empties less efficiently, which leads to a feeling of “fullness”, and the patient's do not feel hungry. Further, with implanting a gastric stimulator, the electrical activity of the stomach can be controlled, regulated, enhanced or competed with.

### Prior Art

U.S. Pat. No. 6,427,089 (Knowlton) is generally directed to using microwave energy to modifying the stomach wall of a patient.

U.S. patent application publication No. 2004/0181178 (Aldrich et al.), application Ser. No. 10/389,236 is generally directed to use of transesophageal delivery of energy to interrupt the function of vagal nerves.

U.S. patent application publication No. 2004/0215180 (Starkbaum et al.), application Ser. No. 10/424,010 is generally directed to ablation of mucosal tissue to inhibit ghrelin production.

U.S. patent application publication No. 2005/0096638 (Starkbaum et al.), application Ser. No. 10/699,207, is generally directed to ablating tissue from an exterior surface of a stomach.

U.S. Pat. No. 6,615,084 (Cigaina) is generally directed to a process of using electrostimulation for treating obesity. An implantable pulse generator (similar to cardiac pacemaker) appears to be used even though details are not provided for stimulation technology.

U.S. Pat. No. 5,423,872 (Cigaina) is also generally directed to a process for treating obesity and syndromes related to motor disorders of the stomach.

U.S. Pat. No. 6,321,124 B1 (Cigaina) is generally directed to the implantable lead aspect of a gastrointestinal pacing system.

### SUMMARY OF THE INVENTION

Method and system for controlling the electrical rhythm of the stomach to provide therapy for obesity, gastric motility, or to induce weight loss comprises ablation of the pacemaker region of the stomach, and implanting a pulse generator. The implanted pulse generator may replace, augment, or interfere with the body's gastric pacemaker function. One of the aims of the therapy is to provide flexibility to alter the rhythmic gastric waves to provide a sustainable form of treatment to achieve the desired results, and avoid side effects like nausea and vomiting among other things which can be associated with abnormal gastric rhythms.

Accordingly, it is one object of the invention, to completely ablate the pacemaker region of the stomach, and implant a pulse generator means to provide electrical rhythm to the stomach.

It is another object of the invention, to partially ablate the pacemaker region of the stomach, and implant a pulse generator to augment or interfere with the residual basic electrical rhythm (BER).

It is another object of the invention, that the combination of ablation and gastric pacing provides the ability to regulate and/or control the gastric rhythm, which can be augmented or inhibited to suit the patient's requirements and needs.

It is another object of the invention, to ablate the pacemaker region of the stomach from the outside (epigastric).

It is another object of the invention, to ablate the pacemaker region of the stomach from the inside (endogastric) via the mouth and esophagus.

In one aspect of the invention, ablations may be performed using Radiofrequency (RF) catheter ablation.

In another aspect of the invention, ablations may be performed with Radiofrequency ablation using irrigated tip catheter.

In another aspect of the invention, ablations may be performed using Microwave ablation.

In another aspect of the invention, ablations may be performed using High intensity focused ultrasound (HIFU) ablation.

In another aspect of the invention, ablations may be performed using Cryoablation.

In another aspect of the invention, ablations may be performed using Laser ablation.

In another aspect of the invention, a programmable implantable pulse generator with a lead comprising electrodes adapted for the gastric muscle may be used.

In another aspect of the invention, a rechargeable implantable pulse generator may be used.

In another aspect of the invention, a stimulus-receiver in conjunction with an external stimulator may be used.

In another aspect of the invention, a dual-channel pulse generator with two leads implanted at different sites may be used.

In yet another aspect of the invention, a dual channel stimulator with two leads may be used, wherein one lead is used for sensing, and the second lead is used for gastric pacing.

This and other objects are provided by one or more of the embodiments described below.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram showing general anatomy of the gastrointestinal (GI) tract.

FIG. 2 is a diagram showing control of the enteric nervous system by the autonomic nervous system (parasympathetic and sympathetic).

FIG. 3 is a diagram showing general anatomy of the human stomach.

FIG. 4 is a diagram showing the longitudinal, circular, and oblique muscle layers of the stomach.

FIG. 5 is a diagram depicting the electrical activity of the GI tract.

FIG. 6 depicts epigastric approach to ablation utilizing laproscopic surgery.

FIG. 7 depicts endogastric approach to ablation via the mouth and esophagus.

FIG. 8A is a simplified block diagram of a radiofrequency ablation system.

FIG. 8B shows a ground patch, and ground patch placement for radiofrequency ablation.

FIG. 9A depicts an area of resistive heating for radiofrequency ablations.

FIG. 9B depicts temperature regions for gastric ablations.

FIG. 10A is a simplified schematic for radiofrequency ablation generator, showing voltage, current, and temperature monitoring.

FIG. 10B is a simplified schematic showing power and impedance as derivatives of voltage and current.

FIG. 11 is a simplified schematic showing the display elements of a radiofrequency ablation generator.

FIG. 12 is a simplified block diagram showing the elements of a microwave ablation system.

FIG. 13 is a diagram showing the principle of high intensity focused ultrasound (HIFU).

FIG. 14 is a simplified block diagram of an ultrasound hyperthermia system.

FIG. 15 is a simplified block diagram of housing for an ultrasound applicator.

FIG. 16 depicts catheter end of an ultrasound ablation system.

FIG. 17 depicts catheter end of a cryoablation probe.

FIG. 18 is cross-section of cryoablation probes.

FIG. 19 is a diagram showing the elements of a laser ablation system.

FIG. 20 is a simplified diagram showing the principle of a laser ablation system.

FIG. 21 is a simplified block diagram showing the elements of a laser ablation system.

FIG. 22 is a simplified diagram of a laser ablation system.

FIG. 23 depicts ablation sites around the pacemaker zone for endogastric approach.

FIG. 24 depicts ablation sites around the pacemaker zone for epigastric approach.

FIG. 25 depicts a gastric pacemaker with electrodes implanted high-up, close to the fundus of the stomach.

FIG. 26 depicts a gastric pacemaker with electrodes implanted close to the lesser curvature of the stomach.

FIG. 27 depicts a dual-channel gastric pulse generator with one pair of electrodes close to the fundus of the stomach, and the other pair close to the lesser curvature of the stomach.

FIG. 28A is a simplified general block diagram of an implantable pulse generator.

FIG. 28B is a diagram of a lead adapted for gastric pacing.

FIG. 29 shows an implantable rechargable pulse generator in block diagram form.

FIG. 30 depicts in block diagram form, the implanted and external components of an implanted rechargable system.

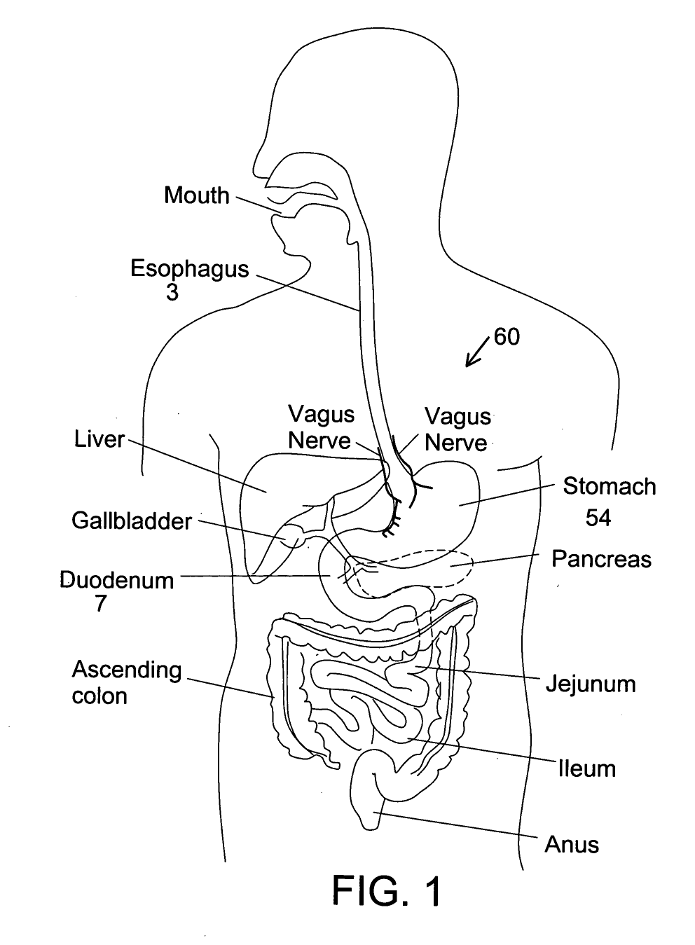

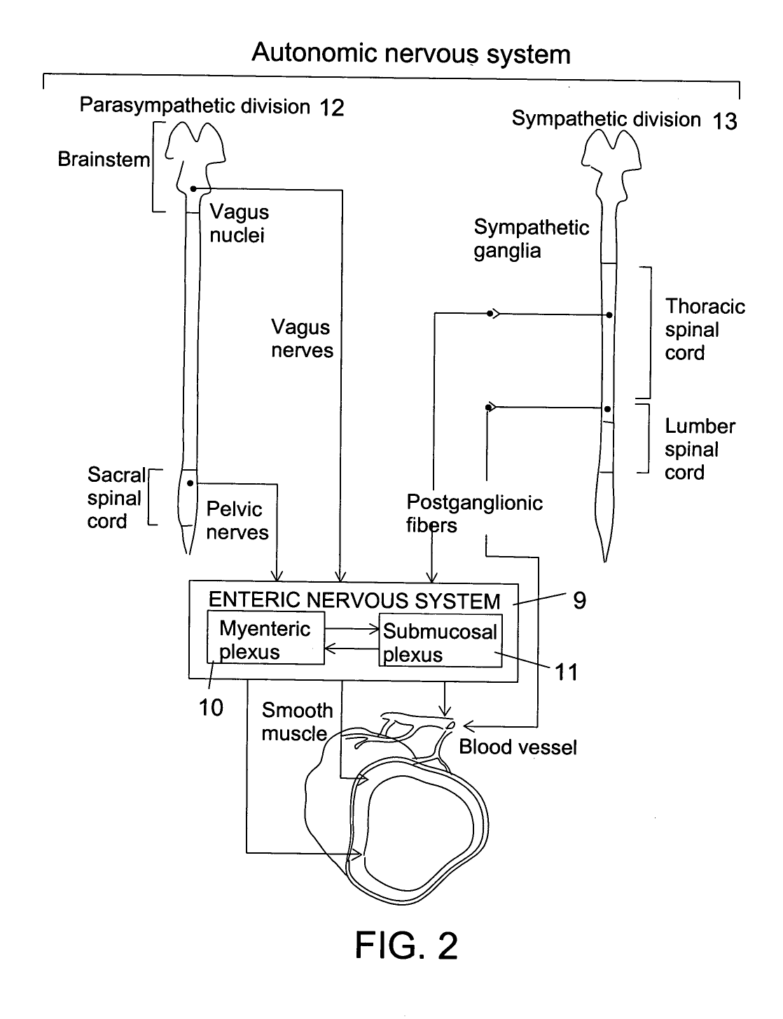

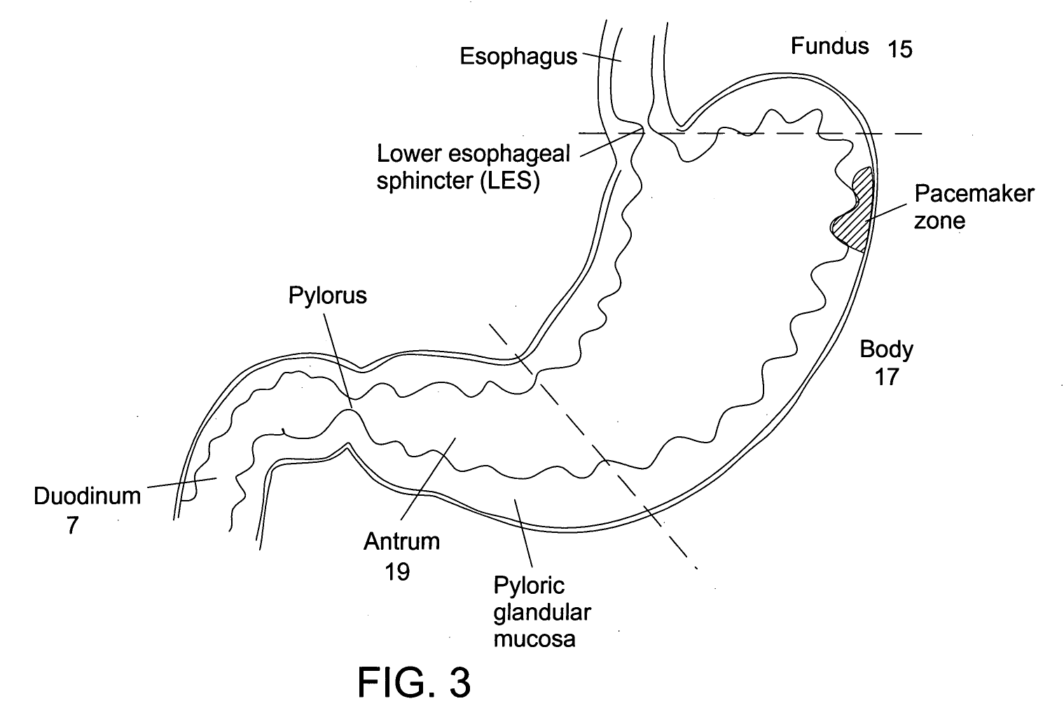

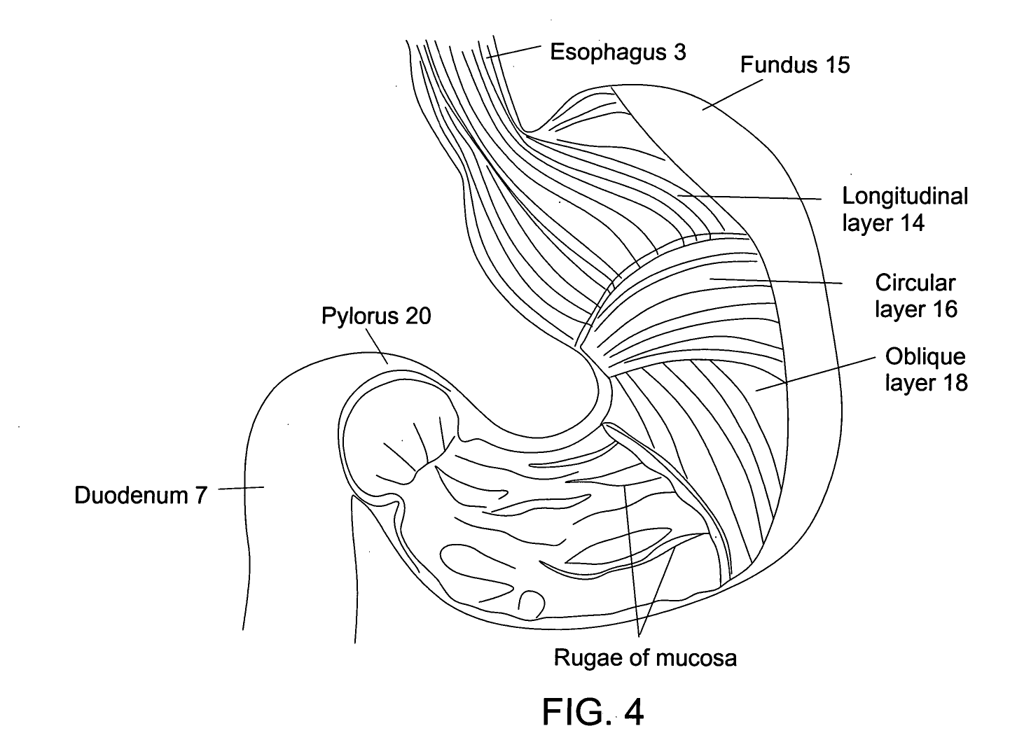

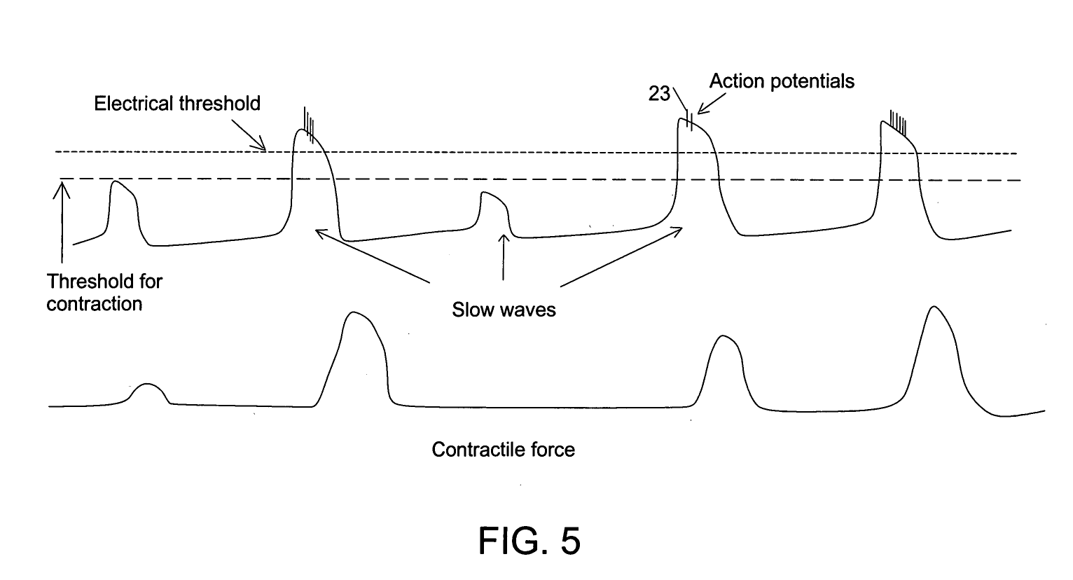

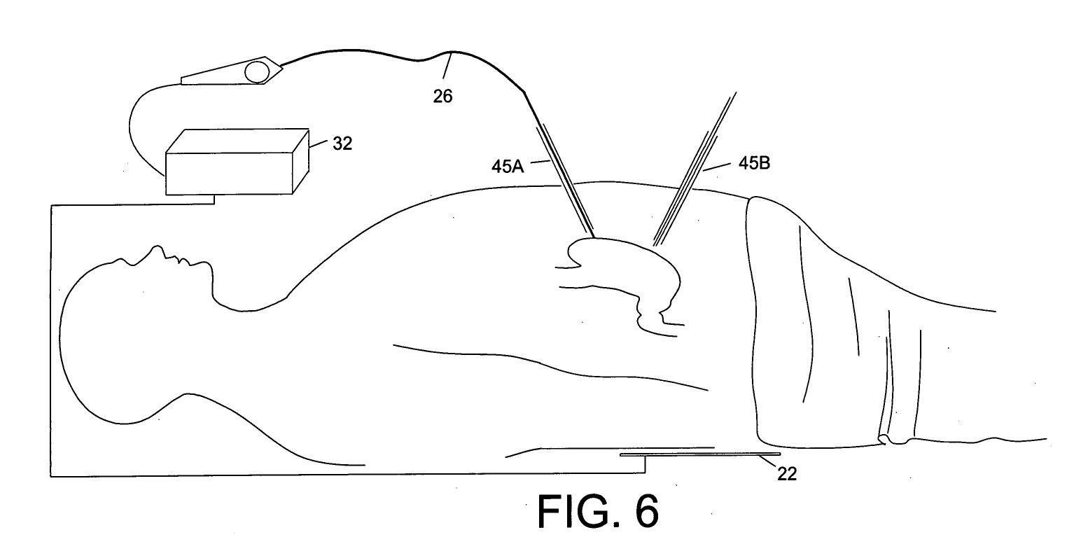

### DESCRIPTION OF THE INVENTION

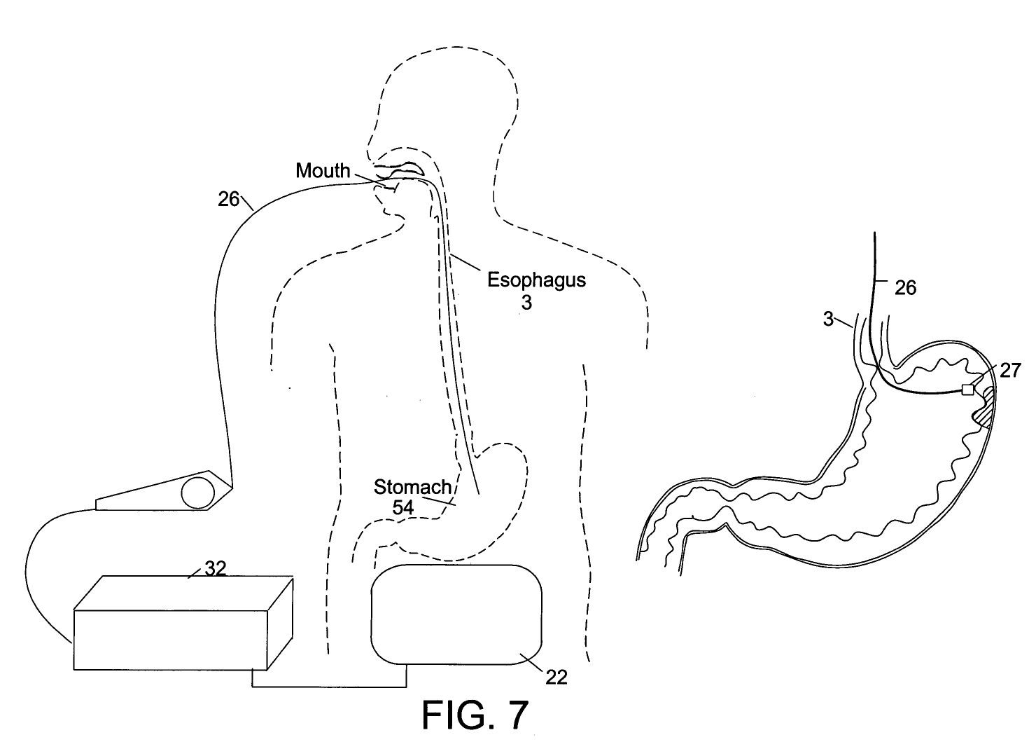

In the method and system of this invention, ablation of pacemaker region of the stomach is performed and a stimulator/pulse generator is implanted to provide therapy for obesity or to induce weight loss. The ablation of stomach may be performed from the epigastric side (shown in FIG. 6) or from endogastric side from within the stomach wall (shown in FIG. 7).

Referring to FIG. 6, for epigastric ablation, the ablation catheter is inserted into the abdominal cavity laproscopically, and ablation lesions are performed on the epigastric surface of the stomach. For performing the ablation procedure, using the epigastric approch, the patient is positioned in the lithotomy position and anesthetized. The abdomen is cleansed with an antiseptic solution and draped in a sterile fashion. The trocars 45A, and 45B are inserted. One trocar 45A is needed for introducing the ablation catheter 26. A second trocar 45B is needed for introducing the optical system. An optional third trocar can be used to introduce a liver retractor.

After retracting the liver, the optical system is used for identifying the anatomical structure to be ablated. Different forms of ablation energies may used, such as radiofrequency (RF) catheter ablation, RF ablation with an irrigated tip catheter, microwave ablation, high intensity focused ultrasound (HIFU) ablation, and cryoablation laser ablation. These are further described later in this disclosure.

Alternatively, the ablation may be performed from the endogastric side (FIG. 7). Combination of epigastric and endogastric ablations may also be performed. As one example without limitation, a patient may have epigastric ablation procedure performed, and at a later date may have endogastric ablation procedure, or vice versa.

As shown in conjunction with FIG. 7, for endogastric ablations, the ablation catheter may be introduced in an anesthetized patient, via the mouth and esophagus, and positioned at the appropriate site within the stomach 54. Even though FIG. 7 is shown in reference to radiofrequency (RF) ablation, other forms of ablation energies may also be used such as RF with irrigated tip catheter, microwave energy, cryoablation, high intensity focused ultrasound (HIFU) ablation, and laser ablation.

In the method and system of this invention, ablation lesions are directed to the pacemaker region of the stomach. The pacemaker region is an area which is close to fundus of the stomach.

Under normal circumstances, the pacesetter cells, which are smooth muscle cells that are capable of rhythmic, autonomous, partial depolariztion, are located in the upper fundus 15 region of the stomach. These cells generate slow-wave potentials that sweep down the length of the stomach toward the pyloric sphincter at a rate of approximately three per minute. Depending on the level of excitability in the smooth muscle, they may initiate contractions recognized as peristaltic waves that sweep over the stomach in pace with the basic electrical rhythm (BER) at a rate of 3/minute. By ablating at, and around the pacemaker region, the intent is to decrease basic electrical rhythm (BER), whereby the stomach empties less efficiently, which leads to a feeling of “fullness”, and the patient's do not feel hungry. Further, with implanting a gastric stimulator, the electrical activity of the stomach can be controlled, regulated, enhanced or competed with.

As described later in this application, the ablations to the pacemaker region may involve complete ablation of the region, or partial ablation to the pacemaker region. In the method of this invention, after the ablation procedure, electrodes are implanted on the gastric wall and an implanted pulse generator (IPG) or stimulus-receiver means are implanted subcutaneously. If the pacemaker region is completely ablated the electrodes are placed closer to the fundus, near the top portion of the stomach. If the pacemaker region is partially ablated, the electrodes are implanted somewhat lower, closer to the lesser curvature of the stomach.

In the method of this invention, the physician uses an interaction of ablations to the stomach and electric pulses to the stomach, to control and regulate the electrical activity of the stomach to provide therapy or induce weight loss.

The ablation technology may be one or more from a group comprising:

a) Radiofrequency catheter ablation;

b) Radiofrequency ablation using irrigated tip catheter;

c) Microwave ablation;

d) Cryoablation;

e) High intensity focused ultrasound (HIFU) ablation; and

f ) Laser ablation.

### Radiofrequency Ablation

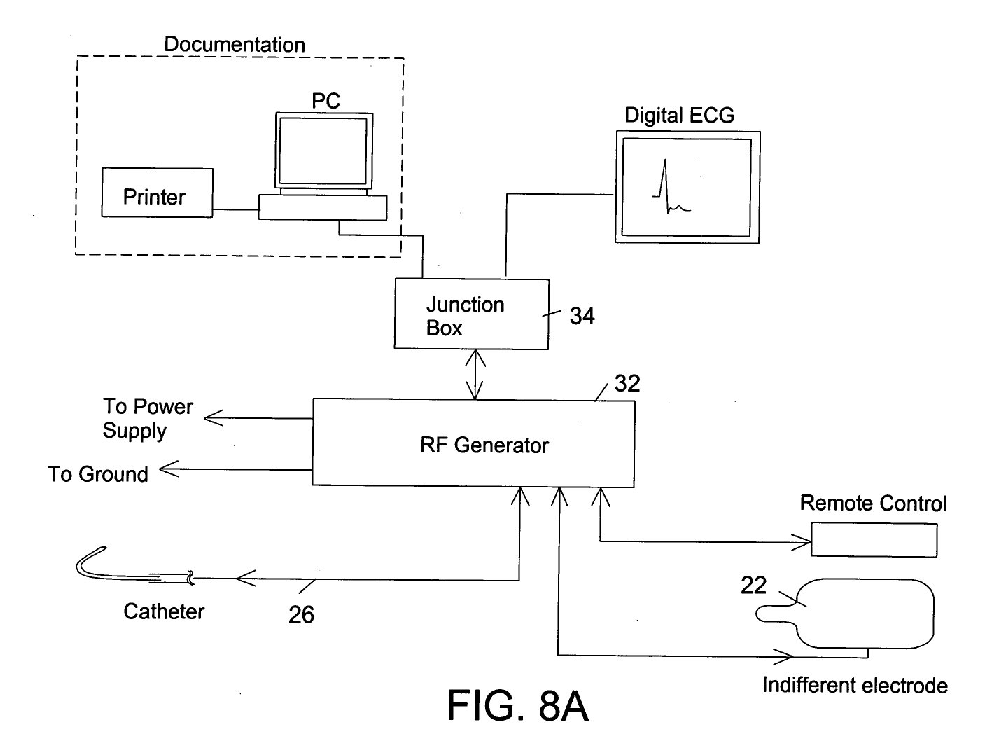

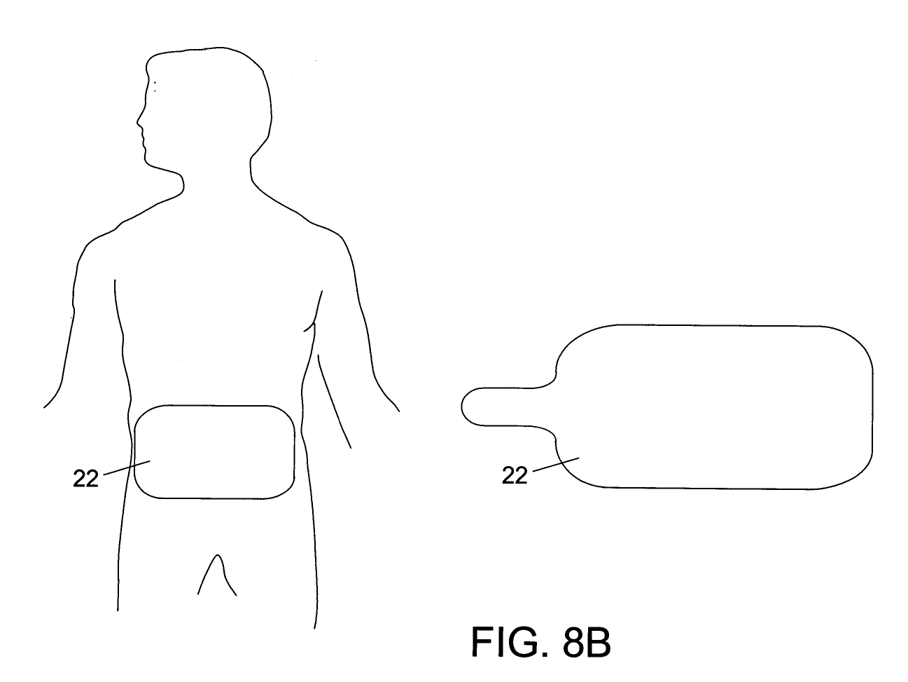

RF ablation is shown with reference to FIGS. 8A and 8B. The RF generator 32 is a source of RF voltage between two electrodes. When the generator is connected to the tissue to be ablated, current will flow through the tissue between the active and dispersive electrodes. The active electrode is placed on for example the stomach tissue where the ablation is to be made, and the dispersive electrode 22 is a large-area electrode forcing a reduction in current density in order to prevent tissue heating. The return electrode (dispersive) (ground pad) 22, is needed to induce resistive heating. Without it the system would look like an open circuit, and there would not be any current flowing through the target tissue and thus there would be no heating. As shown in conjunction with FIG. 8B, the relatively large dispersive electrode 22 would be typically placed on the lower back of a patient, but can be placed at other sites.

When using radiofrequency (RF) ablation, the total RF current, IRF is a function of the applied voltage between the electrodes connected to the tissue, and the tissue conductance. The heating distribution is a function of the current density. The greatest heating takes place in regions of the highest current density, J. The mechanism of tissue heating in the RF range of hundreds of KHz is primarily ionic. The electrical field produces a driving force on the ions in the tissue electrolytes, causing the ions to vibrate at the frequency of operation. The current I density J=σE, where a is the tissue conductivity. The ionic motion and friction heats the tissue, with a heating power per unit volume equal to J2/σ. The equilibrium temperature distribution as a function of distance from the electrode tip, is related to the power deposition, the thermal conductivity of the target tissue, and the heat sink which is a function of blood circulation. The lesion size, is in turn, a function of the volume temperature. Many theoretical models to determine tissue ablation volume as a function of tissue type are available. In RF ablation, lesion formation results from resistive tissue heating at the point of contact with the RF Electrode. This heating leads to coagulation necrosis and permanent tissue damage. If there is poor tissue contact, RF current can not be coupled to the underlying tissue, and the desired effect of tissue heating is lost.

Radiofrequency ablation applies an alternating current to tissue, in the range of 300 to 1 MHz (typically in the 500-KHz frequency range). Unlike direct current, which creates cellular injury via electrolytic dissociation of tissue fluids, alternating current causes tissue damage from heat via protein denatura bon, blood coagulation, and fluid evaporation. It is similar to electrocautery but generally less destructive because of the larger surface area of the surgical probe, and the regulation of power delivery via probe thermistor measurement of tissue temperature.

### Mechanism of Tissue Heating

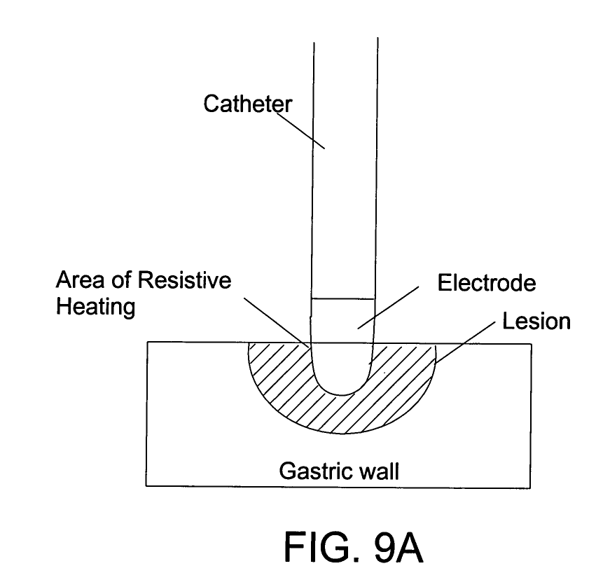

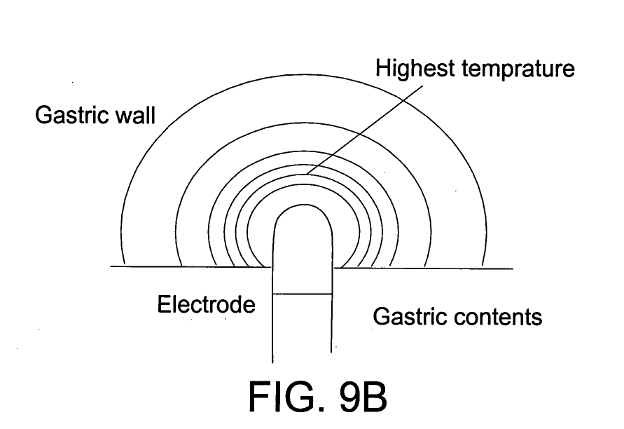

Shown in conjunction with FIGS. 9A and 9B, radiofrequency energy heats tissue in two main ways. First, ohmic heating occurs (FIG. 9A) on the surface by a mechanism in which the gastric tissue in direct contact with the coil or probe acts as a resistor. This heating falls off by the fourth power of distance from the electrode in unipolar systems and typically penetrates only 1 mm. Second, conductive heating occurs (depicted in FIG. 9B), in which this surface heat is transferred to increasingly deeper tissue; conductive heating accounts for the majority of the lesion depth.

RF can be applied either in unipolar (as was depicted in FIGS. 9A and 9B) fashion from a tissue electrode source to grounding pad serving as the indifferent electrode 22, or between two bipolar tissue electrodes. Bipolar RF systems intended for surgical use apply two linear electrodes that gently squeeze together on either side of the gastric tissue. This creates two opposing surfaces of ohmic heating and improves the efficiency with which the conductive heating occurs.

### Determinants of RF Lesion Size

When using RF ablation, the RF electrode temperature is a better predictor of RF lesion size than delivered energy or current. Monitoring of electrode temperature is typically carried out with one or more thermistors. The maximal lesion size from conductive heating is determined primarily by the electrode surface area and electrode-tissue contact temperature, and is achieved at a rate that is a reverse exponential decay with half-time of 7 to 9 seconds.

Lesion size is also influenced by time, irrigation of the electrode, impedance rise, and convective cooling. The duration of energy delivery has a diminishing effect on reaching maximal lesion size after 20 seconds. Electrode irrigation results in deeper lesions. Impedance rises with increased power, increased electrode-tissue pressure, and repeat applications. Saline is protective against impedance rises when compared to blood.

The Bostom Scientific/EP Technologies Cobra system (San Jose, Calif.) is one radiofrequency system approved for commercial use in the United States for general surgical tissue ablation, and may be used for the methods of this invention. The electrosurgical unit (ESU) generates a 500 kHz sine wave. This surgical probe is a flexible single-use probe consisting of seven coagulating electrodes; six of the seven are 12.5 mm coiled electrodes spaced 2 mm apart, and the seventh is an 8 mm distal-tip electrode. Active coils are selected on the ESU prior to the delivery of each lesion. Two skin grounding pads are required to serve as indefferent electrodes.

Finite element simulation of RF ablation using these coil electrodes shows maximal current density at the coil ends, with 2 mm extension of the 50° C. tissue heat isotherm from the coil ends. Each electrode coil contains two temperature-sensing thermistors. One is located 180° apart at each coil end, where resistive heating is greatest. In vitro testing at 80° C. has shown all lesions from adjacent coils to be contiguous, although this is only true in 75% of lesions made at 70° C.

### Electrode and Catheter

To deliver power more efficiently, material which has better thermal conductivity can be chosen. It has been shown that gold, which has four times the thermal conductivity of platinum yields a larger lesion.

The electrode can be designed to cool the tip, thus avoiding tissue charring. The cool-tip catheter using chilled water is one example. Because charring can be avoided. Power can be delivered for a longer time thus allowing the conduction to be carried deeper, thereby increasing the lesion depth. One possible problem with the cool-tip method is the inability to precisely determine the maximum temperature since the maximum temperature is located beyond the cooled electrode surface.

The electrode tip diameter has generally been increased to obtain wider lesions and to allow cooling by nearby fluid flow, thus creating deeper lesions as well. The larger tip diameter, however, creates the need to control nonuniform heating and the presence to hot spots.

Phased RF ablation allows usage of multiple electodes on the same or different catheters. Because adjacent electrodes are in different phases with respect to each other, an RF signal is applied uniformly such that there will be a voltage gradient between electrodes thus creating bipolar heating simultaneously. The advantages of these RF methods include an increase in uniform heating and the possibility to create long, linear lesions, which is useful for gastric muscle lesions.

Balloon electrode RF ablation is another method for a larger tip diameter while still having the ability to be percutaneously inserted. It uses a semipermeable and conductive membrane, such as gold foil, that is inflated with saline when the catheter is inside. Dominant heating occurs at the interface of the balloon and the tissue.

RF electrode design can also use a gel or electrolytic solution, such as saline, instead of direct contact between the metal electrode and the tissue. This produces a more even heat distribution in the tissue. In the design of the electrode for soft tissue shrinkage, the electrolytic solution is cooled to about 30 to 55° C. Not only does this electrolytic solution provide electrical conduction, it also has a cooling effect to avoid too high a temperature at the interface of the electrode and the tissue. Gold coating has been used to prevent corrosion in the saline envirnment. Saline can also be a choice for an irrigation solution because it has the same concentration as the body's fluids, this it is not absorbed by the body.

Having a shaft that can bend 90° can be useful for accessing the back of a joint or the mouth while a bend of 10 to 30° is good for the front part of a joint compartment or the mouth or nose.

### Active Electrode Cooling RF Ablation for obtaining Deeper Lesion

The cooling goal can be obtained with either cool water as in the Cool-tip method or with saline. This method produces a more uniform temperature distribution and allows a longer power delivery thus obtaining a larger lesion without tissue desication.

### Ablation Generator

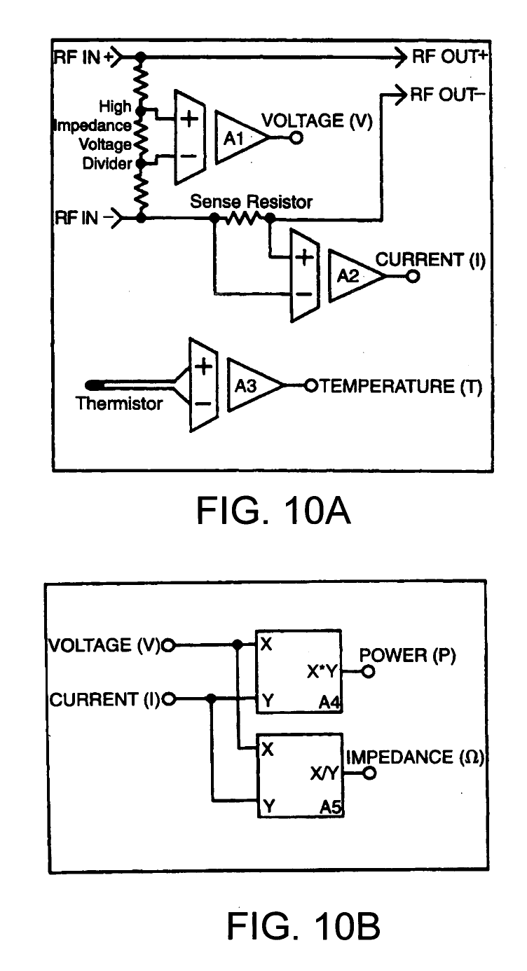

FIGS. 10A and 10B describe in simplified block diagrams the circuitry used to monitor the appropriate radiofrequency parameters. Voltage, current, and temperature are generally measured, and all other parameters are generated. As shown in FIG. 10A, Amplifier A1 uses a high-impedance voltage divider to measure a fraction of the radiofrequency voltage across the outputs. This is isolated, converted to a root-mean-square (RMS) value, and scaled to an appropriate level. The RMS signal has a very-low-frequency waveform and can be easily displayed or digitized at low sampling intervals. Amplifier A2 samples the radiofrequency current by using the current sensing resistor, or a coil can be placed around the return. This signal is also isolated, converted to an RMS value, and scaled. A thermistor is used to measure temperature at the catheter tip. Amplifier A3 isolates the signal, converts the change in resistance to a linearized voltage, and scales the output. The thermistor placement is critical to correct temperature monitoring. This sensor is usually placed as close to the tip as possible and thermally isolated from the rest of the electrode. Even with these precautions, the temperature that is monitored by the system is only an approximation of the tissue temperature at the lesion site. The electrode temperature that is recorded represents a complex interaction of heat generated in the tissue interface, the radiofrequency field, and convective heat loss to surrounding blood and tissue. Although not ideal, it is the best system available.

The signals for power and impedance are derived from the measured values of voltage and current. Given a sinusoidal signal and assuming resistive loads as the major component affecting the output, the following relationships can be used:
Impdence=Voltage/current
Power=Voltage×Current

These associations can be generated by using analog computational blocks as shown in FIG. 10B or by mathematically processing digitized signals.

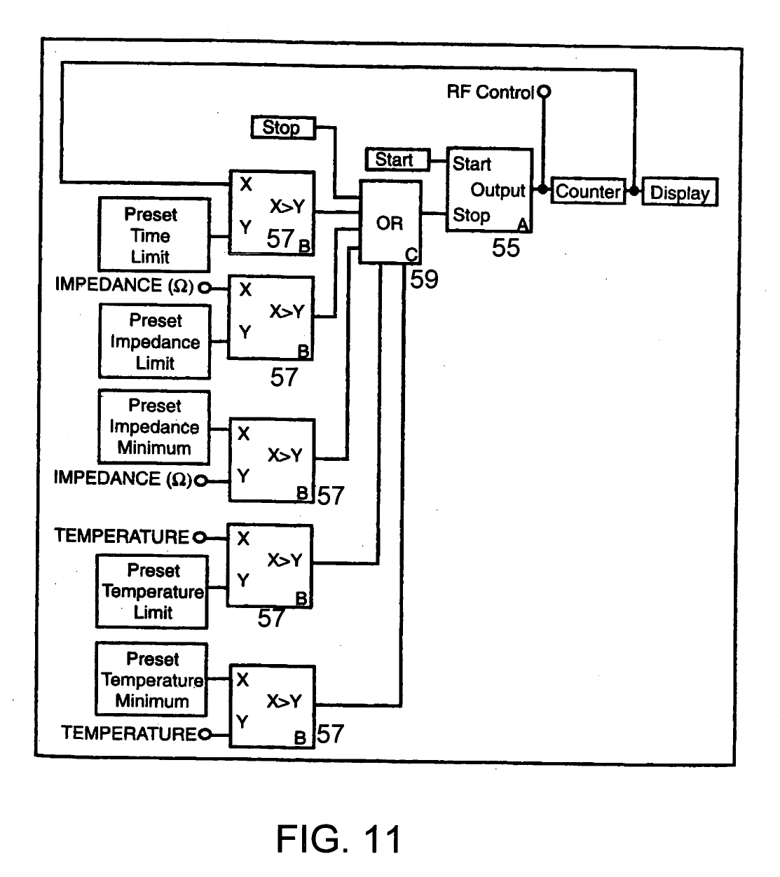

When a generator's output is started or terminated depends on an interaction of the operator and automatic relationships set by the operator or manufacturer. FIG. 11 is a simplified block diagram of a digital control of the generator 32 output. Block A 55 represents a set-reset flip-flop. The output goes true when the start input is set and false when the stop input is set. This output turns on the generator and starts the time counter. Block C 59 is the Boolean OR function and is set true if any of its inputs are true. It serves to sum all of the limit conditions that can stop the generator's output. The B blocks 57 represent comparators, for which the output goes true whenever the X input is greater than the Y input. Otherwise, the output stays false. In this manner, the generator output is terminated whenever the time exceeds the set time, the impedance is outside the set minimum and maximum, the temperature is outside the preset minimum and maximum, or the operator pushes stop Radiofrequency generator can also operate in a power mode. In this mode of operation, time duration is selected, limits on impedance or temperature are set or predetermined by the manufacture, and the desired power level is chosen. The generator outputs the set level of power while allowing the operator to see how the impedance and temperature levels are changing. If an adequate tip temperature is not reached quickly, the operator can terminate the delivered energy or adjust it. If the safety limits of the temperature or impedance settings are exceeded, automatic shutdown occurs.

Because temperature can be crucial to the success of catheter ablation, a temperature mode of operation has been developed. This is also referred to the closed-loop mode of operation. The rationale is to ensure target-tissue temperatures. Instead of the operator choosing a set power level, a temperature set point is selected. The generator then adjusts the power level and monitors the temperature output. Initially, the power is limited as heating begins. The generator then delivers a much larger output level. Usually the maximum, as long as the difference between the set point and the monitored value is larger (10° to 12° C.) than a manufacturer's determined level. After that difference is at or below the manufacturer's setting, power drops off. When the temperature difference becomes sufficiently small (2° to 3° C.), a minimal amount of power is delivered to maintain temperature and to allow monitoring of other parameters. The generators typically cease to deliver power if any of the safety limits are exceeded.

### Microwave Ablation

In the method and system of this invention, microwave energy may be delivered through a probe or catheter antenna to the affected gastric or surrounding tissue which allows the procedure to be performed percutaneously or endoscopically. In microwave ablation, the frequencies 915 MHz and 2.45 GHz are usually used due to Federal Communications Commission (FCC) restrictions.

Unlike RF which generate lesions of relatively limited size and penetration, microwave energy usually allows for greater tissue penetration, and thus a greater volume of heating. Table two below compares some features of RF vs. microwave ablation.

TABLE TWO
Comparison of RF vs. Microwave

| | Radiofrequency | Microwave |
|---|---|---|
| Waveform | Continuous | N/A |
| | Unmodulated sinusoidal | |
| Frequency | 300-1,000 kHz | 915, 2,450 MHz |
| Voltage V | <100 V | N/A |
| Mechanism of injury | Resistive heating | Radiant heating |
| Lesion size | Small | Unknown |
| Control of injury | High | High |

In microwave ablation, a lesion is created as heat conducts passively away from this zone and the surrounding myocardium is heated to a temperature where cell death occurs (approx. 50° C.). Lesion size is therefore a function of the size of the electrode and the resulting temperature at the electrode tissue interface.

The mechanism of thermal injury in microwave ablation is dielectric heating. Body tissue contains various polar molecules, of which water is the most abundant and has an exceptionally high polarity. At microwave frequencies, electromagnetic radiation causes rotation of molecular dipoles; heat is created as these movements are opposed by intermolecular bonds and thus represents dissipation of the part of the energy of the electromagnetic field in the form of molecular friction. Energy absorption is affected by the presence of electrolytes and other polar molecules such as amino acids in tissue water. Conductive heating is a comparatively minor contributor to tissue heating. Heat is produced by the mechanical friction between the water molecules and surrounding structures.

Microwave hyperthermia has shown to be useful in radiation oncology for the treatment of various solid tumors. Also, because of its experience in enlarging myocardial lesions in catheter ablation, microwave energy would be useful in gastric ablation. Microwave energy is delivered down the length of a coaxial cable that terminates in an antenna capable of radiating the energy into tissue. Radiant energy causes the water molecules in myocardial tissue to oscillate, producing tissue heating and cell death. The higher frequency of microwave energy allows for greater tissue penetration and theoretically a greater volume of heating than that possible with RF, which produces direct ohmic or resistive heating.

Microwave energy for tissue ablation effects has been studied using a helical antenna mounted on a coaxial cable (2.44 mm o.d.). High-frequency current at 2,450 MHz was delivered via the helical antenna into a tissue-equivalent phantom model. The temperature distribution profile was measured around the antenna as well as into surrounding volume (the depth of penetration). The volume of heating for the microwave catheter system was 11 times greater than that of an RF electrode catheter at the same surface temperature. In addition, the microwave catheter penetrated an area that was twice as large as that penetrated by the RF catheter. These data suggest microwave energy will produce larger lesion than RF because a greater volume of tissue is being heated, this is advantageous for gastric ablations. An additional theoretical advantage of the microwave system is that direct tissue contact is not crucial for tissue heating since heating occurs via radiation, and not via direct ohmic heating as seen with RF.

Helical and whip antenna designs have also been evaluated in a tissue-equivalent phantom at 915 MHz and 2,450 MHz utilizing a coaxial cable (0.06 in o.d.). All catheters were measured utilizing a network analyzer prior to placing them in the phantom model. Such analysis demonstrated the great variability in tuning of these microwave catheters.

### Microwave Ablation

In general, higher water content (HWC) means higher dielectic loss and HWC tissues will absorb more energy. Low water content (LWC) tissues, such as fat or bone, have dielectric constants and conductivities about one order of magnitude smaller that high water content (HWC) tissues, such as muscle or organs.

Many of the benefits of microwave ablation relate specifically to its mode of heating. Heating occurs in volume and relies very little on thermal flow, allowing microwaves to ablate areas near high blood flow. This is a distinct advantage over RF ablation. Because of the volume heating effect, charring may be eliminated and simply increasing the applied power will also increase lesion size. Power deposition falls as a function of 1/r³ in microwave ablation (as opposed to 1/r⁴ in RF) so power will theoretically travel farther and more uniformly into the tissue. Serious complications apparent in other ablation modalities have not been seen in microwave ablation. Antennas need only be a few centimeters long, reducing the invasiveness of the procedure. Arrays of probes may be employed to increase lesion size or uniformity. In addition, the probe or catheter antennas may be easily sterilized and reused, reducing procedure costs.

### Microwave Generator

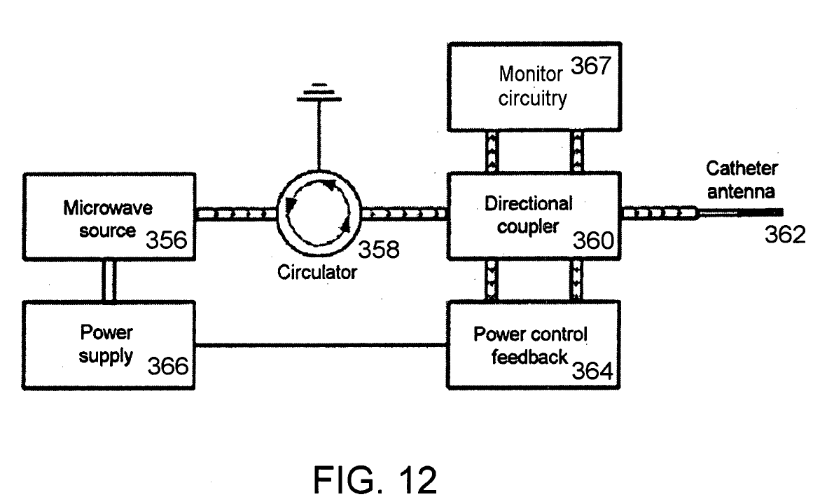

Tissue-ablation microwave generators typically generate the electromagnetic field using a magnetron, such as is used in microwave ovens. The microwave generator provides the necessary microwave power to be delivered to the antenna. Several methods to create this power are available. In general, there are two subcomponents to the generator: a power supply and a microwave source. The power supply converts the line poser (typically 120 VAC, 60 Hz) to a suitable supply for the microwave source. The microwave source then converts the electrical power to microwave power. Shown in conjunction with FIG. 12 is a simplified block diagram of a microwave system, comprising a microwave source 356, coupling network 360, power supply 366, and the catheter antenna 362.

The most common microwave source used in ablation systems is a magnetron due to its low cost, high power output (often several MW), and high conversion efficiency (>80%). The magnetron is a crossed-field resonant cavity tube that converts electron motion to microwave poser. The magnetron filament is heated with a high current (3.3 V, 10 A typical) until thermionic emission causes electrons to “boil” off similar to water molecules boiling off as steam. The high negative potential between the cathode and anode (4 kV typical) creates a large electric field that accelerates the electrons toward the anode. As they accelerate, the axial magnetic field exerts a force on the electrons in a direction perpendicular to their original motion; that is, it pushes the electrons azimuthally around the cathode.

The electric and magnetic field strengths are usually set so that the curving path of an electron just skims the face of the anode block. In this way, the electrons interact with the resonant cavities to set up EM fields. Hence, energy is transferred from the electron motion to the EM fields inside the cavities. Each cavity resonates at the design frequency (2.45 GHz, for example) and a loop is placed inside one of the cavities to extract the microwave power.

The AFx system (AFx, Inc., Freemont, Calif. ) is one currently available microwave system available for cardiac tissue ablation. This system may also be adapted to be used for gastric ablations. The system consists of a magnetron-powered 2.45 GHz generator with power and timer settings, and a hand-held surgical probe that has an antenna at the end through which the electromagnetic radiation is emitted. The Flex-2 is a surgical probe with a 2 cm rigid antenna. The Flex-4 probe has both a bendable shaft and a 4 cm flexible antenna. The antennas have the desirable feature of being shielded on one side. This ensures that only one side of the antenna delivers the ablation energy, an advantage for epigastric ablations.

### High Intensity Focused Ultrasound (Hifu) Ablation

In one aspect of the invention, ablations may be performed using high intensity focused ultrasound (HIFU). When high-intensity ultrasound waves are focused at targets deep within the human body, the temperature in the region of focus can be increased to a level high enough to kill the cells in that region.

Ultrasound has several characteristics which make it well suited for the induction of thermal therapy. These include the feasibility of constructing applicators of virtually any shape and size, and good penetration of ultrasound at frequencies where the wavelengths are on the order of millimeters. The small wavelengths allow the beams to be focused and controlled. Clinical research has shown that ultrasound beams can penetrate deep and that the power deposition pattern can be controlled.

Ultrasound is a form of mechanical energy that is unique among available medical radiation methods in that it can be sharply focused within the tissue. The usual frequency range of medical ultrasound used for imaging and surgical application is 0.5 MHz to 20 MHz. For this range, it has a low absorption rate in soft body tissue and a relatively short wavelength. While the absorption rate limits how deeply the wave can travel inside of the body, the wavelength governs how precisely the wave can be focused onto the tissue. Hence, ultrasonic energy can be deposited deep inside the body with precise focus. As the ultrasound pressure wave travels through the body it loses energy due to scattering and absorption. Scattered energy is used for imaging while energy absorption causes tissue heating.

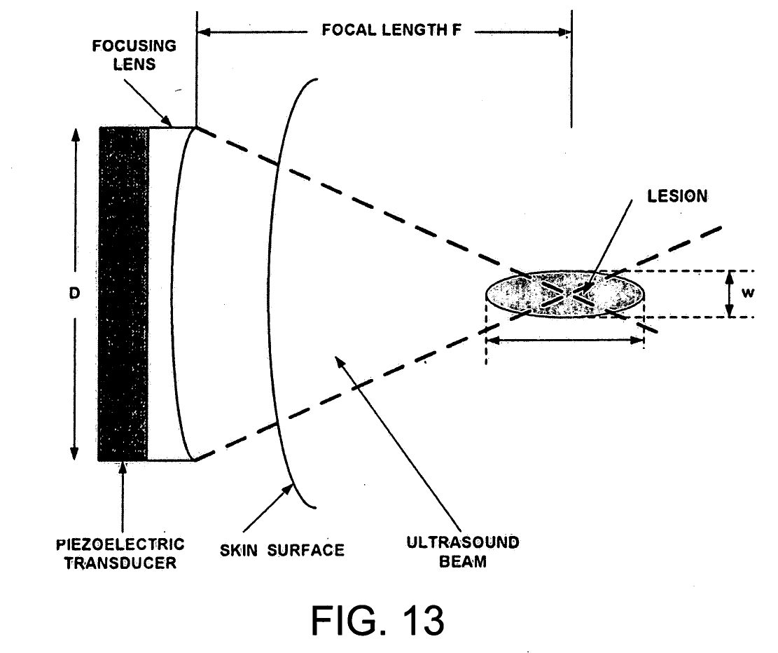

Shown in conjunction with FIG. 13, is the basic principle of the ultrasonic ablation technique which is referred to synonymously as focused ultrasound surgery (FUS) or high-intensity focused ultrasound (HIFU). In this technique, a high-intensity ultrasound beam is brought to a tight focus within the target tissue volume, which may lie deep within the body. The beam passes through the overlying skin and other tissues without harming them. The absorption reaches a maximum in the focal volume where the intensity is at its highest. The temperature at the focal volume is raised to 56 ° C. and held there for 1 to 3 seconds, which kills the cells in focus. There is a very sharp boundary between dead and live cells at the border of the focal volume. Also shown in FIG. 13, the source is a planar ultrasound transducer with diameter D and is situated outside of the body. The ultrasound beam is focused at the desired depth inside of the body by a focusing lens. The lesion produced has length I and width w and is ellipsoid or cigar shaped.

Heating Mechanisms and Biological Effects

HIFU produces an effect on tissues by several mechanisms: thermal effects, cavitation, other mechanical forces, and chemical reactions and acceleration. Thermal and cavitation mechanisms are the most important and best understood. Thermal heating is caused by absorption of ultrasonic energy by the tissues. This leads to a rise in temperature of the tissues. Consequently, the rise in temperature is dependent on the intensity of the ultrasound beam and the heat absorption coefficient of the tissue. In HIFU, the ultrasonic intensity at the beam focus is much higher than that outside of the focus. The ultrasonic focus can easily generate temperature elevation of 30° C. to 40° C., coagulating tissue in just a few seconds.

### Ultrasonic Ablation System

A complete HIFU system would normally consist of an ultrasonic applicator, electromechanical components for steering and positioning the acoustic beam, a display for therapy planning and imaging, and a computer for HIFU dosage calculations and control, as well as, for monitoring feedback during ablation.

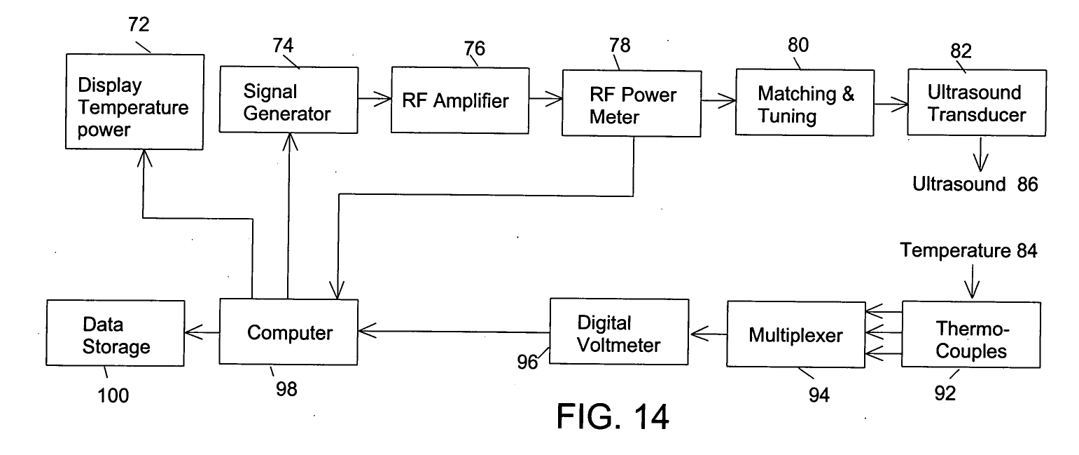

Shown in conjunction with FIG. 14 is a simplified block diagram of an ultrasound system for hyperthermia induction. The RF signal is generated by a signal generator 74 or an oscillator and is amplified by an RF amplifier 76. The generation of the RF signals to be converated into mechanical motion is in principle similar in all systems. The forward and reflected electrical power are measured after amplification in order to obtain the total acoustic power output. The signal enters the transducer through a matching and tuning 80 network that couples the electrical impedance of the transducer 82 to the output impedance of the power amplifier 76. The power output is controlled by the amplitude and duty cycle of the RF voltage.

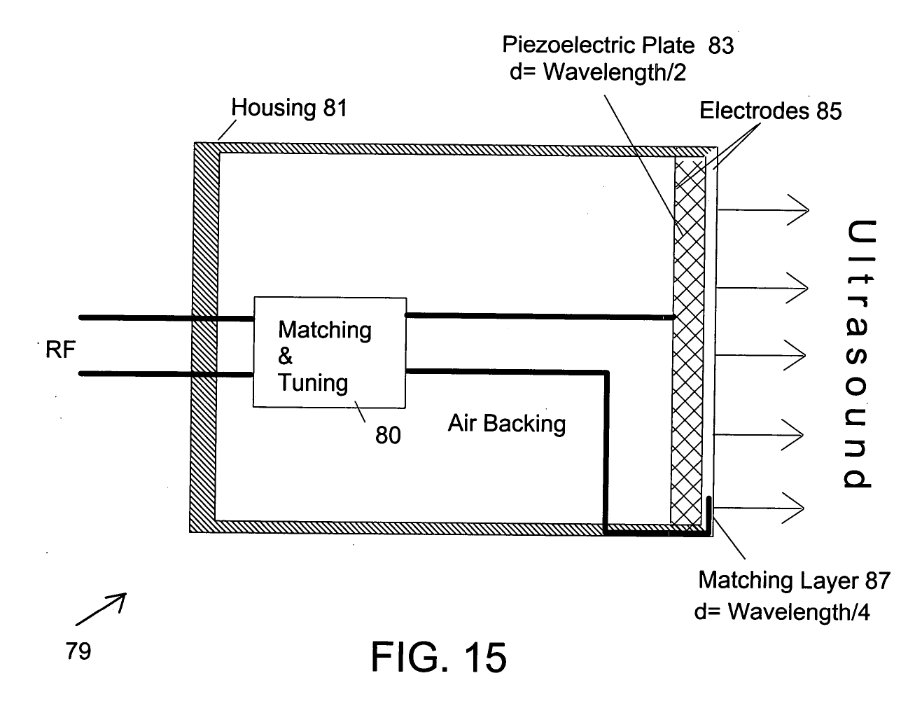

Shown in conjunction with FIG. 15 is a general structure of a high-power ultrasound transducer. The thickness of the plate of piezoelectric material 83 determines the operating frequency. Both surfaces of the transducer are covered by thin metal electrodes 85. The transducer plate is mounted on the holder in such a way that it has maximum freedom to move. On the front surface there can be a one-quarter wavelength matching layer 87 that reduces the acoustic mismatch between the transducer and the coupling media. However, it is optional and adequate power outputs can be obtained without it. An air space behind the plate provides a low impedance backing. This space can also house the electrical matching circuit 80. Maximum electrical efficiency of the transducer can be obtained when the transducer is matched to the electrical impedance of the driving amplifier and the electrical and mechanical resonances of the transducer are tuned together.

Piezoelectric materials lack a center of symmetry in their lattice structure, and have the property that the application of pressure causes an electrical voltage to appear across the crystal. The voltage is proportional to the applied pressure within the elastic limits of the material. By applying a changing voltage across a piezoelectric crystal, electrical energy can also be converted to mechanical thickness change of the crystal. As is known in the art, since hyperthermia transducers capable of producing high power, single-frequency continuous waves for extensive periods are needed, lead zirconate titante (PZT) is generally used. Also in reference to FIG. 15, the maximum stress wave is obtained when the thickness of the plate d=λ/2 or an odd multiple of λ/2. The frequency which corresponds to the half wavelength thickness is the fundamental resonant frequency of the transducer.

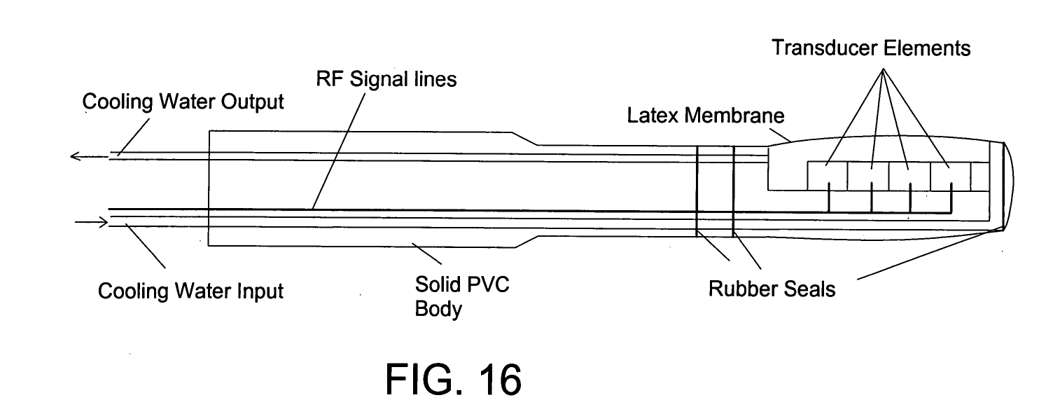

For the application of the current invention, the piezoelectric ceramic can be manufactured in the shape of a cylinder with electrodes on both inner and outer surfaces. When an RF voltage is applied on the electrodes, the cylinder wall thickness will expand and contract with the voltage. This generates a cylindrical ultrasound wave which propagates radially outward. Cylindrical applicators are known in the art for delivering for prostate applications, and can be similarly used for gastrointestinal (GI) applications of the current invention. One such four-element intracavitary applicator is shown in conjunction with FIG. 16. As will be clear to one of ordinary skill in the art, these can be adapted for the various gastric applications.

### Cryoablation

Cryoablation generally is a surgical technique that employs freezing to kill the target cells. The target tissue is frozen to a lethal temperature dependent on the tissue type to generate an ice ball. Accurate monitoring of the ice ball margin and temperature is achieved by employing intraoperative ultrasound and placing thermocouples inside of the cryoprobe.

The mechanism of tissue injury in cryoablation are not fully understood and there are some controversies about then. Generally, two mechanisms are considered as the main causes of direct cellular injury: (1) cell dehydration by osmosis when the ice ball is created in the extracellular space, and (2) intracellular ice formation at a high cooling rate.

At slow rates of cooling, tissues tend to freeze extracellularly. Slow cooling rates encourage the crystals to expand to a very large size. When these crystals develop in the extracellular space, migration of water out of the cells occurs because of the pressure gradients induced by the combined influence of concentration differences and capillarity. The ultimate end of such a process is dehydration of the cells and the development of external ice crystals which can be many times the size of individual cells.

At high cooling rate, the migration of water out of the cells may become inadequate to support the rapid growth of extracellular crystals. As a consequence, intracellular ice formation occurs, probably from growth of external ice through minute water-filled pores in the cell membrane. Intracellular ice crystals will tear down the membranes of cells and organelles inside the cell.

### Cryogen

Liquid nitrogen and argon are widely used as cryogens. The boiling temperatures of LN₂ and argon are −196° C. and −186° C., respectively. However, this low temperature is hard to attain in the probe design. One reason is back pressure, which limits the flow of cryogen into the cryoprobe, and the other reason is Liedenfrost boiling.

### Cryoprobe

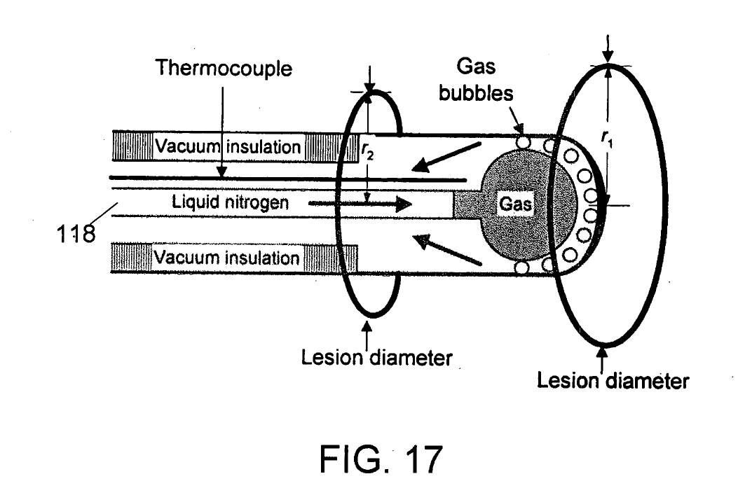

The LN₂ probe generally consists of a closed-end tube with two tubes concentrically arranged within it. Shown in conjunction with FIG. 17, is the basic design for a typical LN₂-based cryoprobe. Inside the probe, there is a funnel 118 for liquid nitrogen to go through. At the end of funnel, it hits the warm uninsulated tip of the cryoprobe where it changes phase, expanding 700 times in volume. The expanding gas exits the cryoprobe around the supply tube. This gas expansion is the constraint on the probe's functioning since it creates a back pressure that limits the flow of liquid nitrogen into the cryoprobe. Another phenomenon, caused by phase change, is the Liedenfrost boiling. When liquid nitrogen expands, gas bubbles form between the liquid and the metal, acting as an insulator. As a result, the temperature of the cryoprobe tip is about −160° C., not −196° C. The rate of complications and adverse effects are significantly higher with LN₂-based systems due to a slow response time to control adjustment.

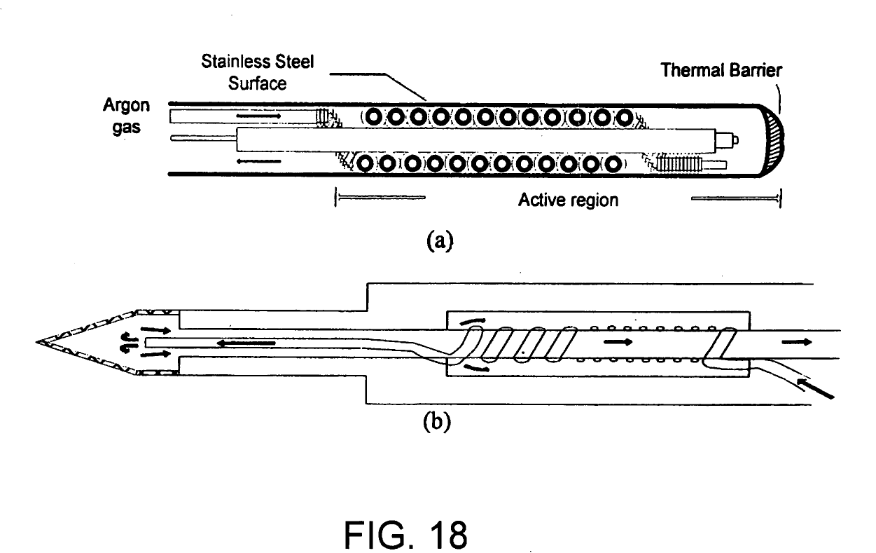

Shown in conjunction with FIG. 18 is an argon-based cryoprobe available from Endocare Inc., in which the system operation is based on the Joule-Thomson principle. Such a system can also be adapted for gastric ablation. In this system when a gas flows from a region of higher pressure to a region of lower pressure through a constricted passage (J-T port), it is said to be throttled. Based on Joule and Kelvin's principles, we know that most gases drop in temperature when throttled. For some gases, notably hydrogen and helium, the temperature rises. Whether there is a rise or fall in temperature depends on the particular range of pressures and temperatures over which the change occurs. For each gas, there are different values of pressure and temperature at which no temperature change occurs during a Joule-Thomson expansion. That temperature is the inversion temperature. The ratio of the observed drop in temperature to the drop in pressure is the Joule-Thomson coefficient (dT/dP). The temperature of a particular gas increases or decreases after going through a J-T port depending on whether its original temperature is above or below its maximum inversion temperature. Generally, the temperature decreases as long as the maximum inversion temperature is above ambient temperature and vice versa.

FIG. 18(b) shows a different type of cryoprobe available from Galil Medical Ltd. The details of these systems are disclosed in U.S. Pat. No. 5,800,787 (a) and U.S. Pat. No. 6,142,991 (b), which are incorporated herein by reference.

### Laser Ablation

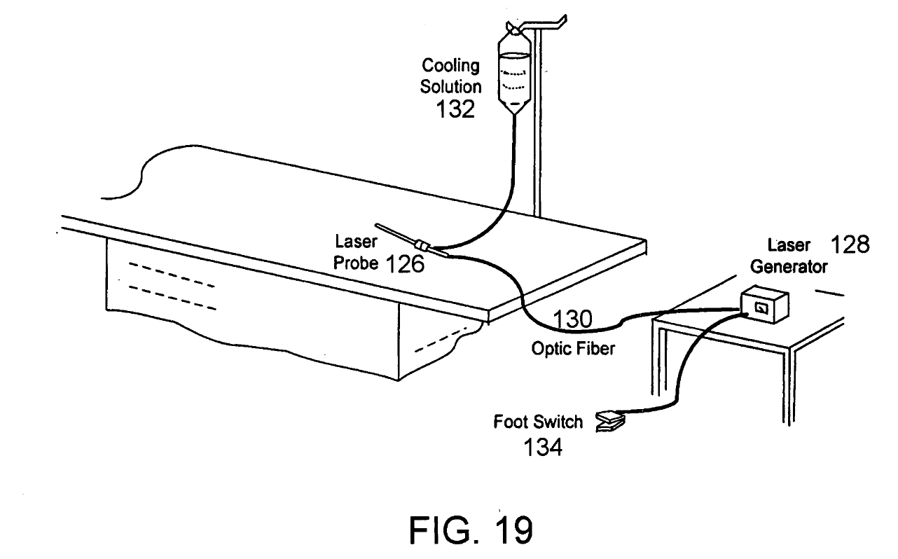

Lasers are widely used in ablations and many other medical applications, and can be adapted for use with gastric or other gastrointestinal (GI) ablations of the current invention. Lasers are coherent, and the energy of a laser beam is concentrated in a very narrow wavelength band. All photons in a laser beam are exactly in the same phase. Lasers are always directional. The direction of a laser beam is exactly parallel to the axis of the laser generator cavity. Lasers have these properties because of the way lasers are generated A typical laser ablation system is shown in conjunction with FIG. 19. The system consists of a solid state laser generator 128 with control system, an optical fiber cable 130, a laser probe 126, a water cooling system 132, and an external foot switch 134.

### Laser Tissue Ablation

With laser ablation, tissues are ablated through tissue coagulation, water vaporization, tissue dehydration, tissue cabonization and pyrolysis. Ablated tissue can be directly removed through vaporization and explosive mechanical ruptures.

Laser System Lasers are generated inside laser generator resonate cavities. The lasing medium could be a gas, dye, solid state crystal, or semiconductor. The excitation mechanism converts the electric power from the power supply unit to other types of energy to excite the lasing medium. After the lasing medium is excited, its molecules are energized from their low energy levels to their higher energy levels, which is the inverted population state. The lasing medium in its inverted population state emits free photons when its molecules transit from their higher energy levels back to their low energy levels. When free photons travel in the lasing medium and pass by other excited molecules, the excited molecules are stimulated to transit from their higher energy levels to their lower energy levels causing them to emit photons of the same frequency, phase, and direction as the free photons. This is the phenomenon of stimulated emission. Free photons are amplified by the stimulated emission effect in the laser generator cavity in all directions. Most of them will quickly exit from the cavity if they are not moving in a direction exactly parallel to the axis of the lasing cavity, and will be reflected back and forth between the high reflector and the output coupler, which is in fact, a partial reflector. Photons in the cavity-axis direction are reflected between the two reflectors. They are amplified by the stimulated emission effect of the excited lasing medium. Amplified photons form the unique phased and unidirectional output laser beam.

Compared to the complete reflector at one end of the cavity, the output coupler at the other end is actually a partial reflector. It lets the amplified laser photons partially exit from the laser cavity to become the output laser beam. The majority of the laser photons remain in the cavity to be further amplified by the excited lasing medium. The output coupler is usually connected with other optical delivery devices such as an optical fiber cable, which will conduct the output laser beam to the tissue where the laser beam will be applied.

The excitation mechanism excites the lasing medium and keeps it at its inverted population state. The excited lasing medium amplifies the laser beam in the cavity through the stimulation emission effect. The whole laser cavity is a balanced laser system as the electric power is taken from the power supply unit and converted to the output laser energy by the excitation mechanism and the lasing medium.

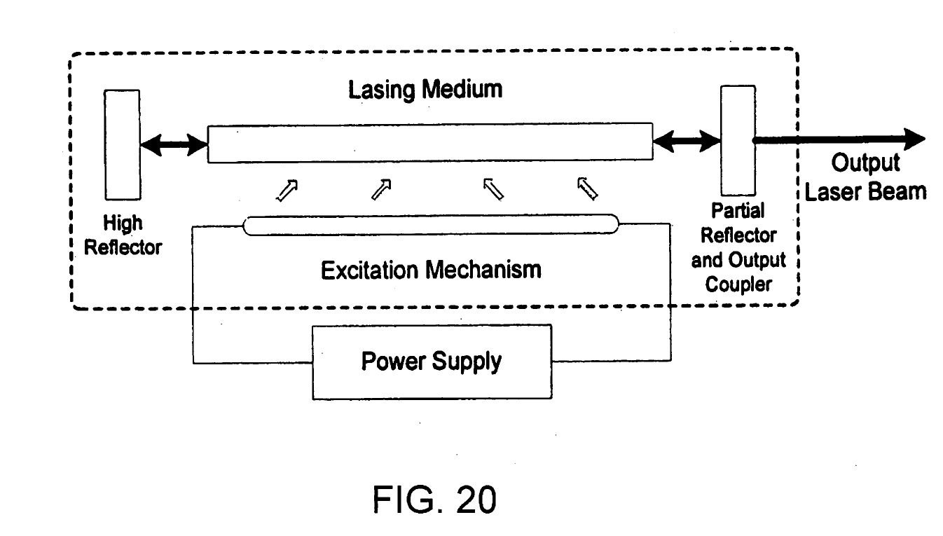

FIG. 20 shows a schematic diagram of a working laser generator, which can be adapted for gastric ablation application. The excitation mechanism is powered by the electric power supply unit and excites the lasing medium. Photons are amplified by the excited lasing medium and resonate between the high reflector and the output coupler. The output coupler releases a certain amount of laser photons to form the output laser beam.

### Laser Ablation Systems

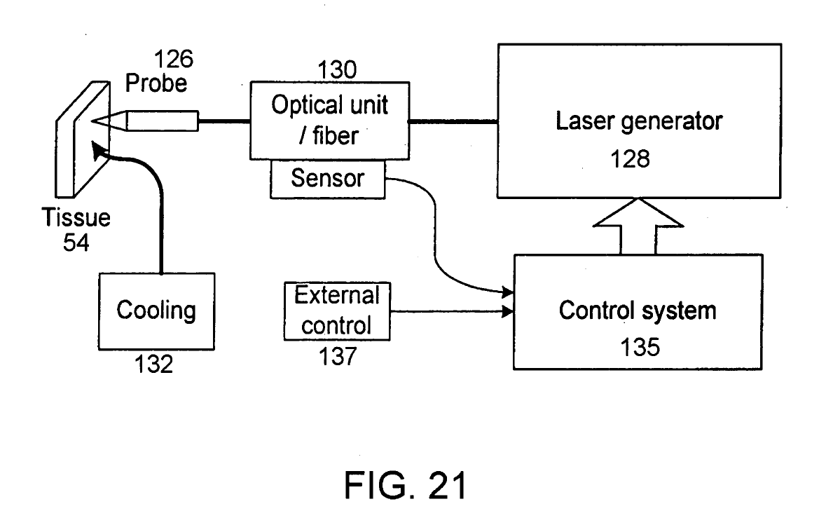

As shown in conjunction with FIG. 21, laser ablation systems usually consist of laser generator 128, computerized control systems 135, optical conductive units including optical fiber cables 130, laser probes 126. Lasers are generated by laser generators 128, which are controlled by their control systems 135. Output laser beams are coupled into optical delivery systems or optical fiber 130 cables and conducted to the laser probes 126. The laser probes then apply the laser beams to target tissues, which is the pacemaker region of the stomach 54.

### Control Systems

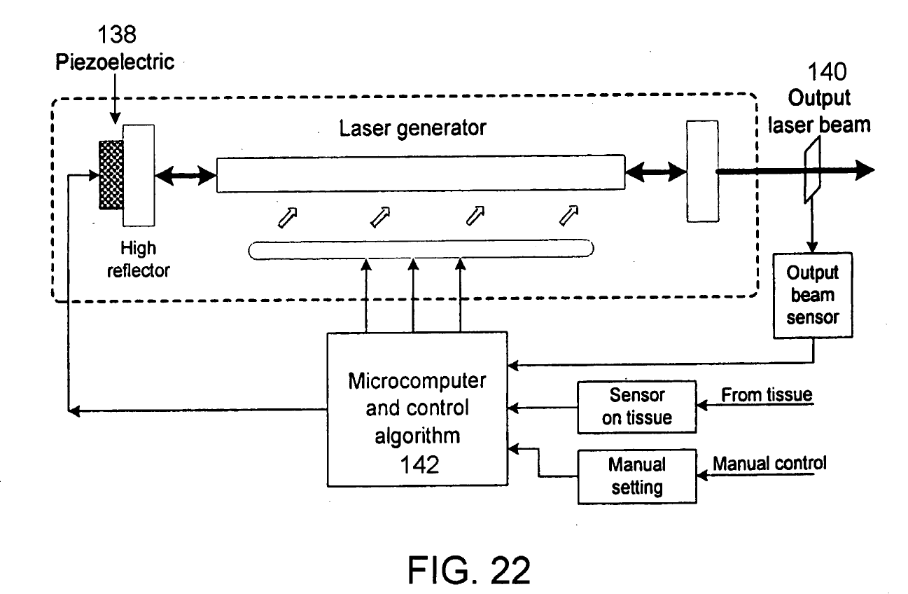

Control systems usually vary among different laser ablation systems. They are essential in controlling laser ablation procedures. Shown in conjunction with FIG. 22 is a generic control system. The microcomputer 142 with control algorithm is the center. The control center integrates inputs from different sensors and manual control settings, calculates optimized parameters by using these inputs according to preprogrammed control algorithms, and controls its effectors on the laser generator-the reflectors and the excitation mechanism. The generic control system shown in FIG. 22 can control laser output, pulse duration, and pulse frequencies by controlling the piezoelectic transducer 138, and it can control the laser output 140 pulse power densities by controlling the pumping mechanism.

The cooling systems effectively remove the heat which is generated when laser radiation energies are absorbed by target tissues. They reduce the amount of heat transferred to adjacent tissues, minimize the damage to the adjacent tissures, and improve the precision of ablation procedures. Both air spraying and water spraying are used as cooling mechanisms.

As is well known in the art, these systems can be adapted for gastric or other gastrointestinal (GI) tract ablations of the current invention.

### Tissue Ablation and Stimulator Implant

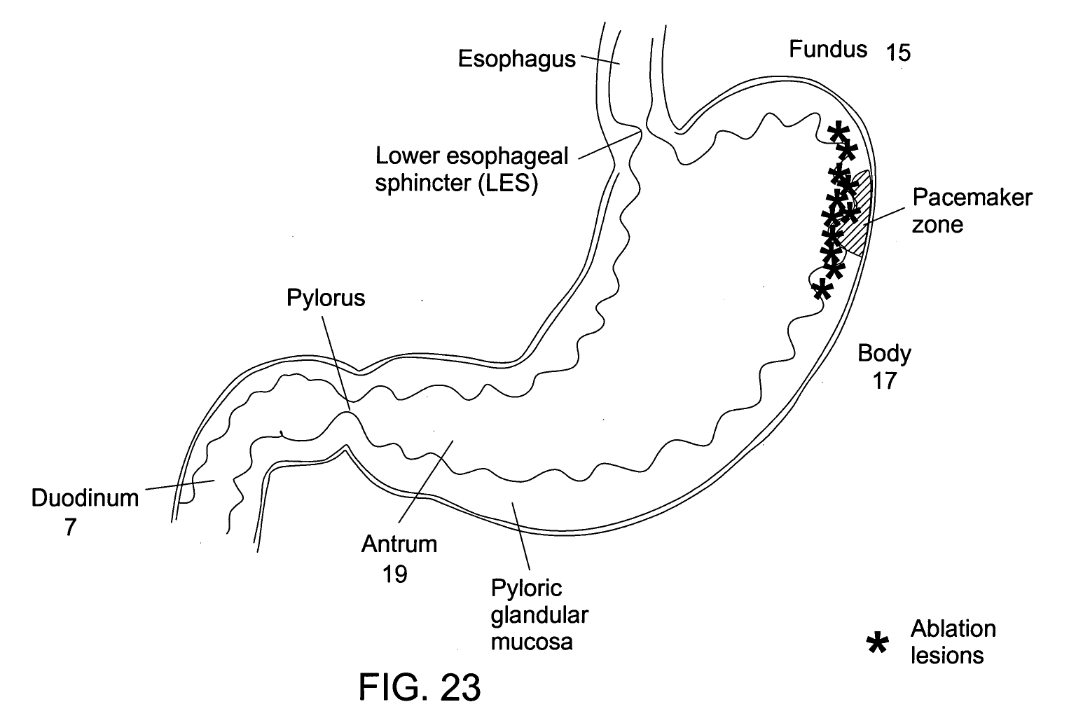

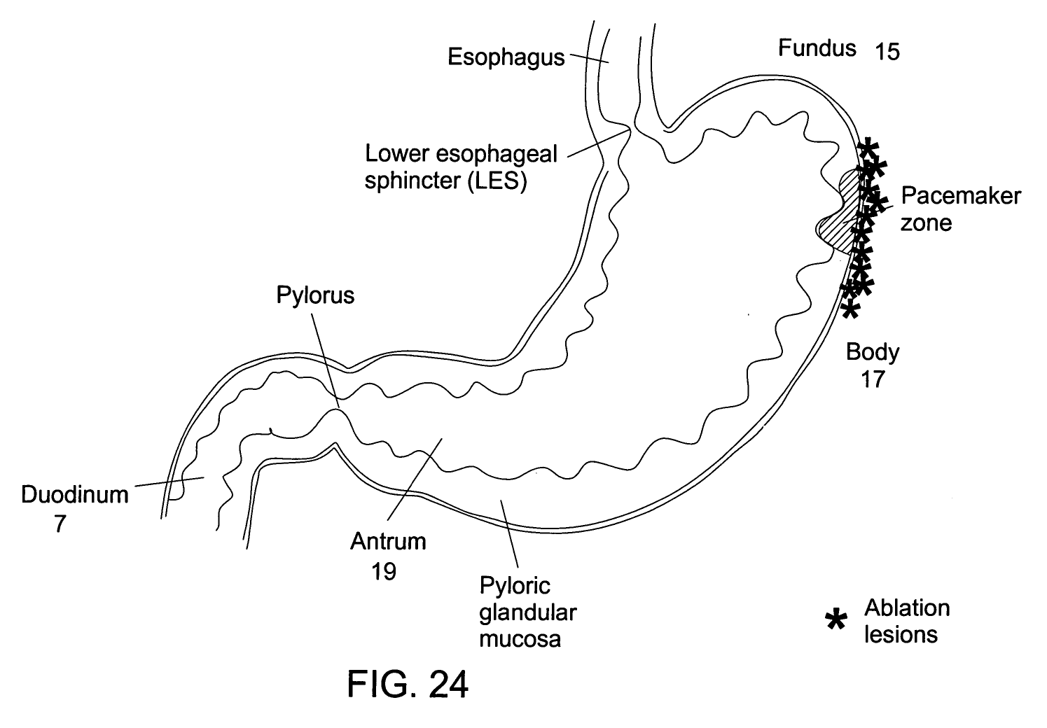

According to one object of the invention, shown in conjunction with FIGS. 23 and 24 are sites marked which are around the approximate area for ablation lesions. The ablations are at the pacemaker zone of the stomach, which is an area close to the fundus 15 of the stomach. As was mentioned previously, the ablations may be from the epigastric side via laproscopic surgery, or may be via endogastric side through the mouth and esophagus. FIG. 23 depicts lesions from the endogastric side, and FIG. 24 depicts lesions from the epigastric side. The ablation technology used may be one from the following:

a) Radiofrequency catheter ablation;

b) Radiofrequency ablation using irrigated tip catheter;

c) Microwave ablation;

d) Cryoablation;

e) High intensity focused ultrasound (HIFU) ablation; and

f) Laser ablation.

The number of lesions or the amount of ablation performed is dependent upon the physician, and the type of ablation technology used. In another object of the invention partial destruction of the pacemaker zone of the stomach may be performed, and a stimulator (pulse generator) may be used to augment the basic electrical rhythm (BER) as needed to optimize the objectives of the therapy. It is another object of the invention, to completely ablate the pacemaker zone and have the patient's gastric rhythm be dependent upon an artificial stimulator. The combination of ablation and electrical pulses via an implantable stimulator can control and/or regulate the electrical activity of the stomach. It will be clear to one skilled in the art, that any amount of interaction between the level of ablation and level of gastric pacing can be achieved, and such judgement is at the discretion of the physician based on the therapy goals, or the amount of weight loss desired.

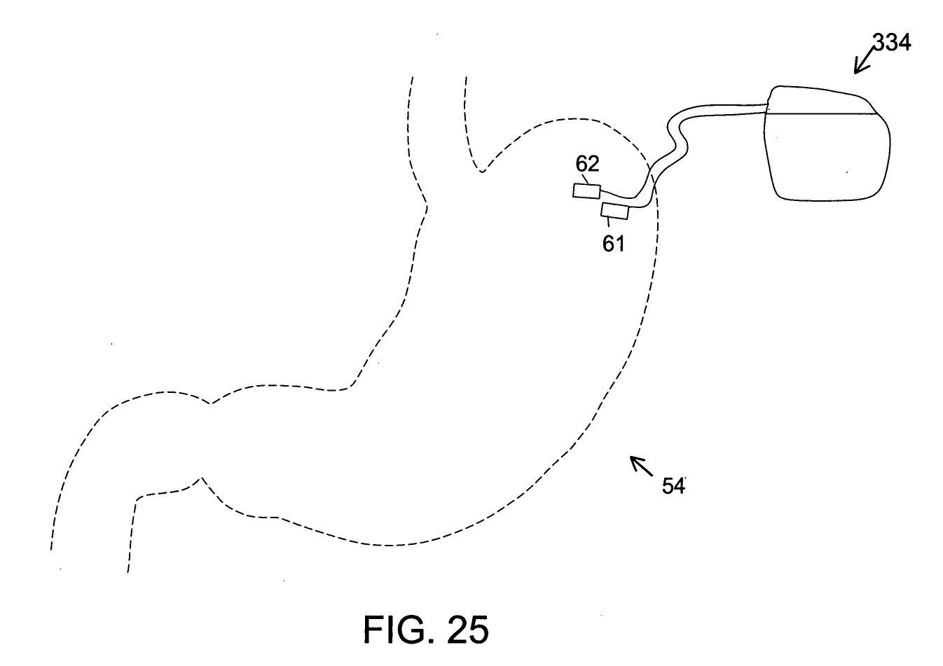

Further, the anatomical placement of pacing electrodes on the stomach 54 is also at the discretion of the physician. FIG. 25 depicts placement of electrodes 61,62, high up on the stomach 54, close to the fundus 15 region of the stomach 54. This would be appropriate if complete ablation of the pacemaker region has been performed, and the patient has little or no intrinsic rhythm left. In this case, the aim of the stimulation is to replace the intrinsic rhythm, and the rate of peristatic waves can be fully controlled, depending on the desired results.

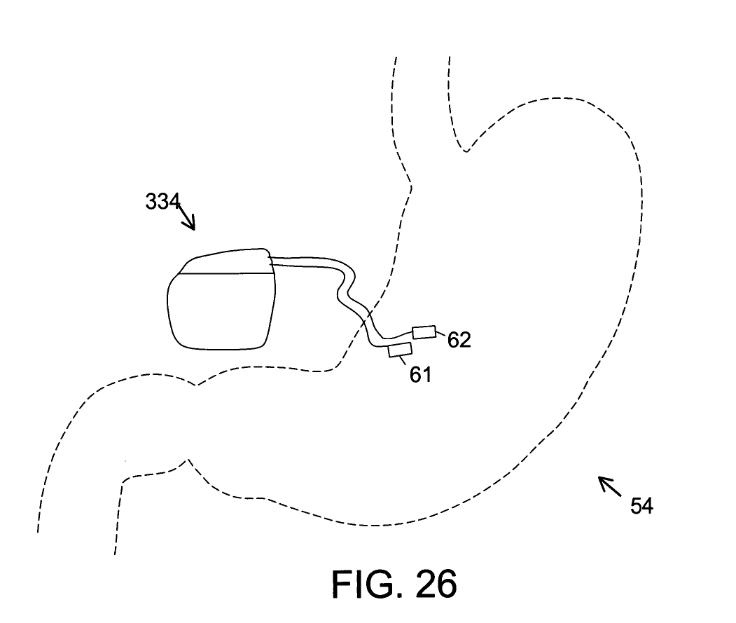

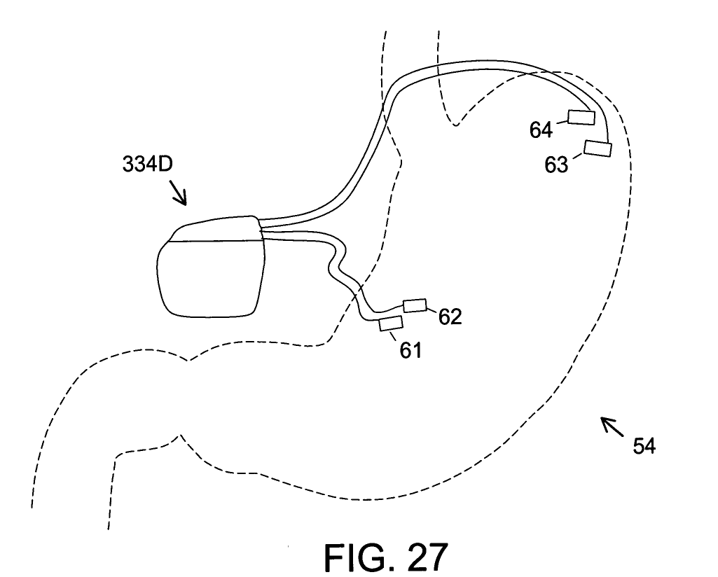

Shown in conjunction with FIG. 26, is a depiction where the stimulation electrodes 61,62 are implanted lower, closer to the lesser curvature of the stomach 54. This is more appropriate, if partial ablation of the pacemaker region is performed, and the patient still has a certain level of basic electrical rhythm (BER). In one preferred embodiment of the invention, two pairs of electrodes 61,62,63,64 (using two leads) may be implanted on the stomach 54. This is shown in conjunction with FIG. 27. In this embodiment, one pair of electrode 63,64 is implanted relatively high-up on the stomach close to the fundus 15 of the stomach 54. The second pair of electrodes 61,62 are implanted at a lower site, closer to the lesser curvature of the stomach. In this case, the two leads are connected to a dual-channel stimulator 334D. Dual-channel stimulators are well known in the art. As will be clear to one of ordinary skill in the art, the physician could non-invasively program via a programmer (not shown) which of the leads to use for stimulation, as well as, program the other electrical parameters such as pacing rate, pulse amplitude and pulse width etc.

Furthermore, in one preferred embodiment one pair of electrode can be used for sensing, and the other pair can be used for gastric pacing. Sensing will provide an index for the effects of ablation, and the level of intrinsic gastric activity present. It will be clear that a dual-channel stimulator provides the physician with a lot of flexibility, especially since the ablation procedure can performed in multiple gradual steps.

The gastric stimulator may be an implantable pulse generator (IPG), a rechargeable IPG, or an implanted stimulus-receiver designed to function in conjunction with an external stimulator.

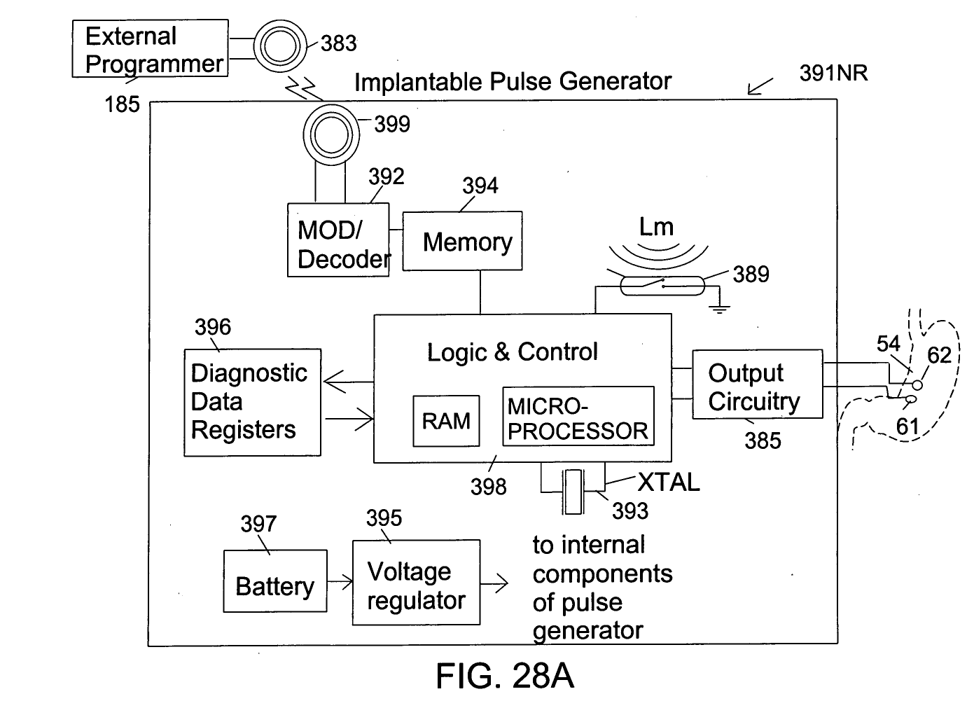

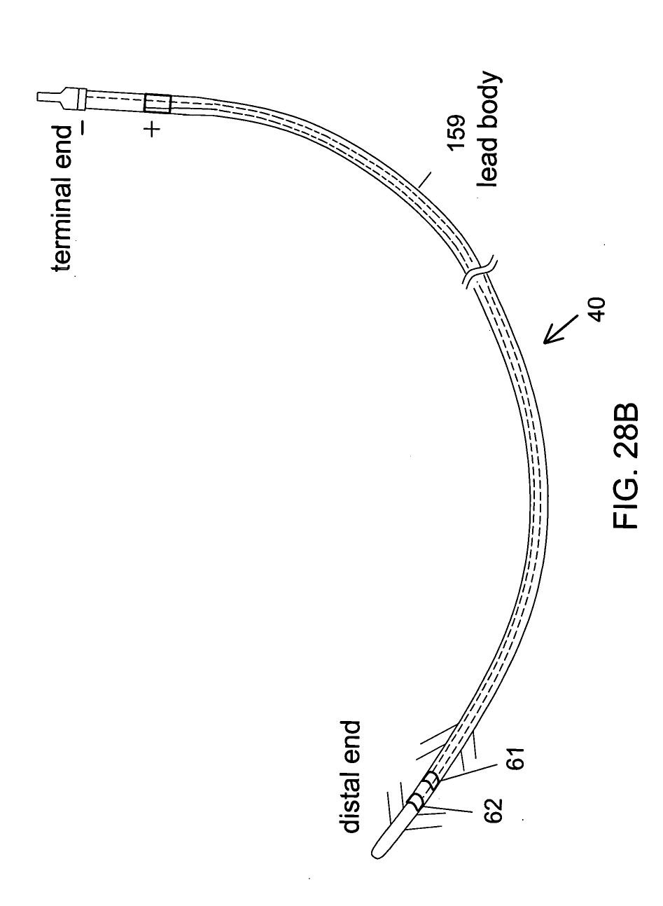

The IPG is preferably a multi-programmable microprocessor based device, as shown in conjunction with FIG. 28A. The implantable pulse generator unit 391NR is a microprocessor based device, where the entire circuitry is encased in a hermetically sealed titanium case. As shown in the overall block diagram, the logic & control unit 398 provides the proper timing for the output circuitry 385 to generate electrical pulses that are delivered to a pair of electrodes 61, 62 via a lead 40 (depicted in FIG. 28B). Timing is provided by a crystal oscillator 393. The pair of electrodes to which the stimulation energy is delivered is switchable in the dual-channel stimulator (not shown). Programming of the implantable pulse generator (IPG) is done via an external programmer 185. Once programmed via an external programmer 185, the implanted pulse generator 391 NR provides appropriate electrical stimulation pulses to the gastric wall 54 via the stimulating electrode pair 61,62. In this disclosure, the terms stomach, stomach muscle, gastric wall, and gastric wall muscle are used interchangeably.

Implanted pulse generators such as used in cardiac pacing or nerve stimulation may also be adapted for gastric pacing. Therefore, implanted pulse generators available from Medtronic Inc. (Minn., Minn.), Cyberonics Inc. (Houston, Tex.), Transneuronix Inc. (N.J.), and others may be adapted for the methodology of this invention, and are incorporated herein by reference.

Because of the high energy requirements for the pulses required for stimulating the gastric wall muscle 54 (unlike cardiac pacing), there is a real need for power sources that provide an acceptable service life under conditions of continuous delivery of pulses. Accordingly in one aspect of the invention, the implantable pulse generator (IPG) may be a rechargeable IPG.

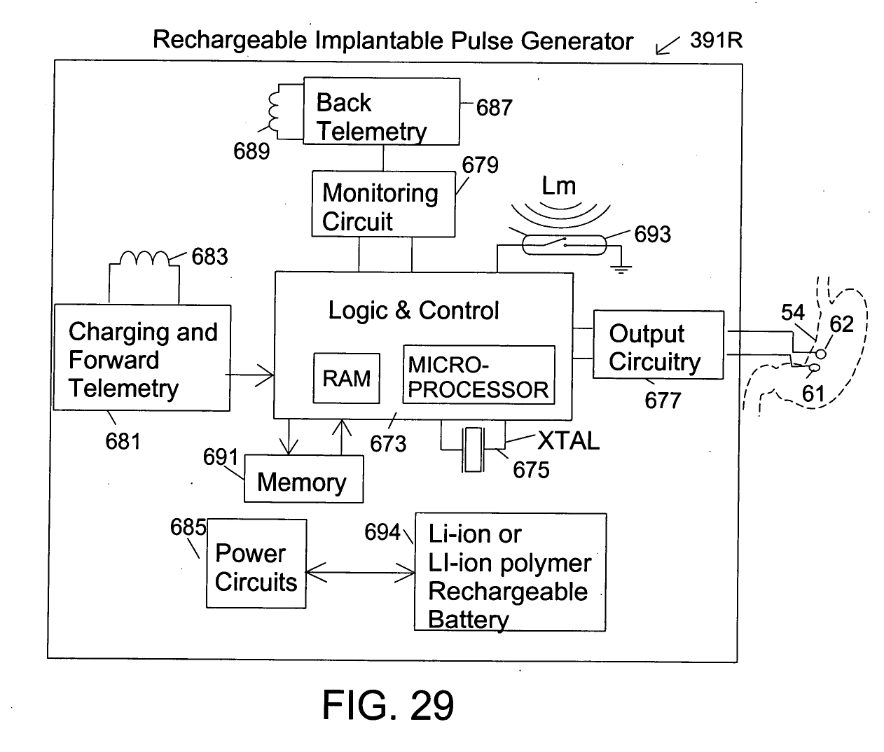

Applicant's co-pending application Ser. No. 11/047,233, entitled “Method and system for providing electrical pulses to gastric wall of a patient with rechargeable implantable pulse generator for treating or controlling obesity and eating disorders”, discloses a rechargeable IPG for gastric wall stimulation, this application is incorporated herein, in its entirety, by reference. The salient features are summarized here for reader convenience. Shown in conjunction with FIG. 29 is a schematic diagram of the implanted pulse generator (IPG 391R) with re-chargeable battery 694 of one preferred embodiment. The IPG 391R includes logic and control circuitry 673 connected to memory circuitry 691. The operating program and stimulation parameters are typically stored within the memory 691 via forward telemetry. Stimulation pulses are provided to the gastric muscle wall 54 via output circuitry 677 controlled by the microcontroller.

The operating power for the IPG 391R is derived from a rechargeable power source 694. The rechargeable power source comprises a rechargeable lithium-ion or lithium-ion polymer battery 694. Recharging occurs inductively from an external charger to an implanted coil 48B underneath the skin. The rechargeable battery 694 may be recharged repeatedly as needed. Additionally, the IPG 391R is able to monitor and telemeter the status of its rechargable battery 694 each time a communication link is established with the external programmer 185.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from titanium and is shaped in a rounded case.

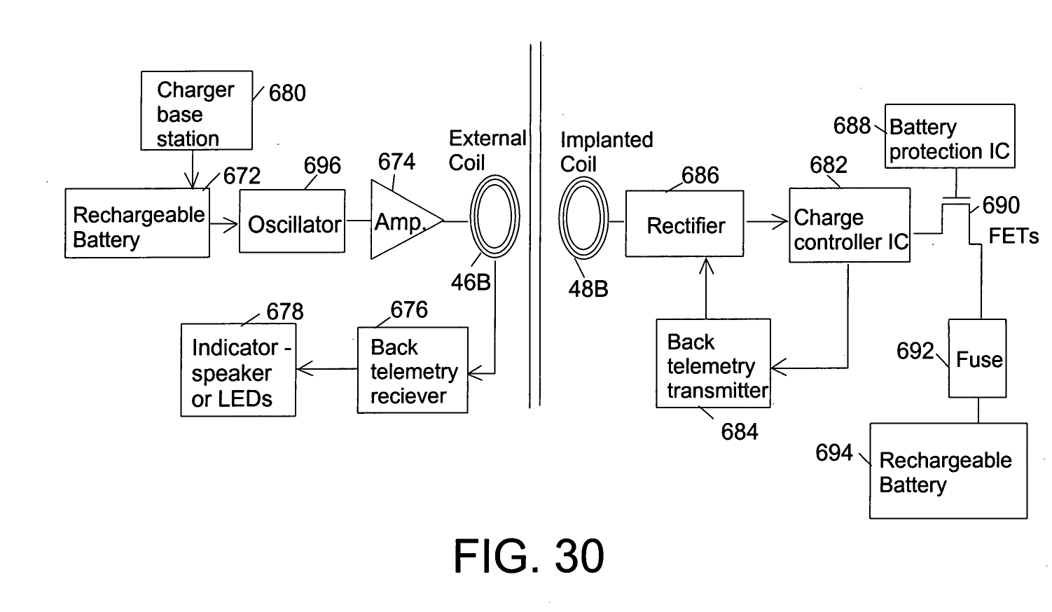

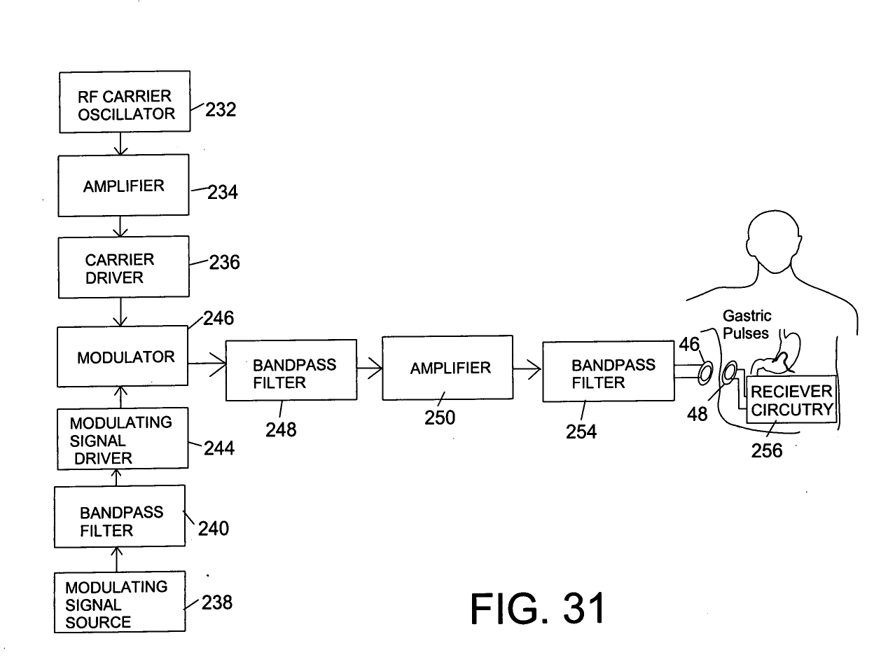

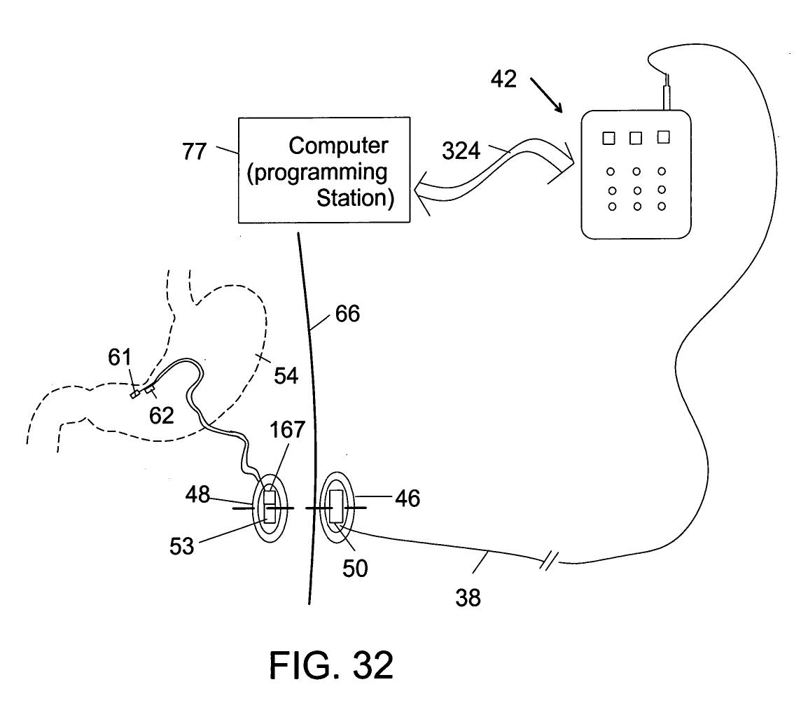

Shown in conjunction with FIG. 30 are the recharging elements. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 30, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

In one aspect of the invention, gastric pacing may be performed with an implanted stimulus-receiver used in conjunction with an external stimulator. Such an inductively coupled system is disclosed in applicant's co-pending application Ser. No. 11/032,298 entitled “Method and system for providing electrical pulses to gastric wall of a patient with an external stimulator for treating or controlling obesity and eating disorders”. This application is also incorporated herein, in its entirety, by reference.

It is summarized here for reader convenience in conjunction with FIGS. 31 and 32. FIG. 31 shows, in block diagram form the delivery methodology to deliver pulses to the gastric wall. A modulator 246 receives analog (sine wave) high frequency “carrier” signal and modulating signal. The modulating signal can be multilevel digital, binary, or even an analog signal. In this embodiment, mostly multilevel digital type i.e., pulse amplitude and pulse width modulated signals are used. The modulated signal is conditioned 248, amplified 250 and transmitted via a primary coil 46, which is external to the body. Shown in conjunction with FIGS. 31 and 32, a secondary coil 48 of the implanted stimulus-receiver, receives, demodulates, and delivers these pulses to the gastric wall 54 via electrodes 61 and 62.

Many embodiments of the invention have been described. Various modifications may be made without departing from the scope of the claims. It is therefore desired that the present embodiment be considered in all aspects as illustrative and not restrictive, reference being made to the appended claims rather than to the foregoing description to indicate the scope of the invention.

1. A method of treating at least one of obesity, motility disorders or to induce weight loss, comprising the steps of ablating a pacemaker region of the stomach and providing electrical pulses to the stomach for controlling and/or regulating the electric activity of said stomach.

2. A method for at least one of controlling, regulating, slowing, enhancing the basic electrical rhythm (BER) of the stomach muscle, for treating or alleviating the symptoms of at least one of obesity, motility disorders or to induce weight loss, comprising the steps of:
providing ablation to a pacemaker region of said stomach; and
providing electrical pulses with a pulse generator for pacing said stomach for controlling and/or regulating the electrical activity of said stomach muscle.

3. The method of claim 2, wherein said ablation is provided by at least one of radiofrequency (RF) catheter ablation, RF ablation using irrigated tip catheter, microwave ablation, high intensity focused ultrasound ablation (HIFU), cryoablation, or laser ablation.

4. The method of claim 2, wherein said ablations to a pacemaker region may further be epigastric and/or endogastric ablations.

5. The method of claim 2, wherein said RF ablation is delivered with frequencies ranging from approximately from 300 KHz to 1,000 KHz.

6. The method of claim 2, wherein said microwave ablation is performed at approximately at 945 MHz, or approximately at 2,450 MHz.

7. The method of claim 2, wherein said electrical pulses maybe provided for enhancing and/or countering the ablation effect.

8. The method of claim 2, wherein said electrical pulses are provided by a pulse generator which is one from a group comprising, a programmable implantable pulse generator, a rechargeable implantable pulse generator, an external stimulator used in conjunction with an implanted stimulus-receiver.

9. The method of claim 8, wherein said implantable pulse generator is a two-channel pulse generator capable of functioning with two leads.

10. The method of claim 8, wherein said pulse generator is further connected with a lead assembly with at least one electrode adapted to be secured to said stomach muscle for providing said electrical pulses.

11. The method of claim 2, wherein said electrical pulses are provided at one or more site(s) anywhere on said stomach muscle.

12. The method of claim 2, wherein said electrical pulses provided further comprise variable parameters which are programmable over a predetermined range of values to effectively treat said at least one of obesity, motility disorders or to induce weight loss.

13. The method of claim 2, wherein said electrical pulses comprise amplitude between 0.5 volt and 25 volts, pulse width between 5 milliseconds to 2 seconds, and pulse rate between 1 cycle/min. to 100 cycles/min.

14. A method of ablating and pacing the stomach with electrical pulses to treat or alleviate the symptoms of at least one of obesity, motility disorders or to induce weight loss, comprising the steps of:
providing ablation to a pacemaker region of said stomach by at least one of radiofrequency (RF) catheter ablation, RF ablation using irrigated tip catheter, microwave ablation, high intensity focused ultrasound ablation (HIFU), cryoablation, or laser ablation; and
implanting an implantable stimulator/generator device adapted to provide predetermined electrical output signal upon activation of said device, and an implantable electrical lead assembly connected to said implantable stimulator/generator device, and with at least one electrode adapted to be secured to said stomach muscle for electrical excitation of the stomach muscle to modulate the electrical activity of said stomach muscle.

15. The method of claim 14, wherein said ablation of pacemaker region may further be epigastric ablation and/or endogastric ablation.

16. The method of claim 14, wherein at least one ablation lesion is provided to the pacemaker region.

17. The method of claim 14, wherein said RF ablation is delivered with frequencies ranging from approximately from 300 KHz to 1,000 KHz.

18. The method of claim 14, wherein said implantable stimulator/generator device is a dual channel generator, wherein at least one lead is capable of sensing the intrinsic gastric activity.

19. The method of claim 14, wherein said electrical pulses comprise amplitude between 0.5 volt and 25 volts, pulse width between 5 milliseconds to 2 seconds, and pulse rate between 1 cycle/min. to 100 cycles/min.

20. The method of claim 14, wherein said implantable stimulator is a dual channel stimulator comprising two implantable leads.
